(19) United States
(12) Patent Application Publication
(10) Pub. No.: US 2003/0228682 A1
(43) Pub. Date: Dec. 11, 2003

(54) Fluorescence sensing

(75) Inventors: Joseph R. Lakowicz, Ellicott City, MD (US); Chris D. Geddes, Bel-Air, MD (US)

Correspondence Address: SUGHRUE MION, PLLC, 2100 Pennsylvania Avenue, NW, Washington, DC, 20037-3213, US

(73) Assignee: University of Maryland, Baltimore

(21) Appl. No.: 10/426,012

(22) Filed: Apr. 30, 2003

Related U.S. Application Data
(60) Provisional application No. 60/376,967, filed on Apr. 30, 2002.
(60) Provisional application No. 60/416,112, filed on Oct. 4, 2002.

(51) Int. Cl. C12M 1/34; G01N 33/553

(52) U.S. Cl. 435/287.2; 436/525

(57) ABSTRACT

An apparatus and a method for detecting or measuring the presence of a compound capable of fluorescing in a sample. The Apparatus may contain a metal particle and a compound capable of fluorescing separated by at least one film spacer layer. The thickness of the film enhances the fluorescence of said compound due to the distance of the compound from the metal particle. The method includes spacing the compound at a distance from a metal particle, which provides an enhanced fluorescence intensity of the compound, exposing the compound to radiation and detecting the fluorescent emission. The Apparatus may also be in the form of multiple metal particles in a porous three dimensional matrix. The method also includes flowing the compound through a porous three dimensional matrix comprising multiple metal particles.

### CROSS REFERENCE TO RELATED APPICATIONS

This application claims benefit of priority of U.S. provisional application No. 60/376,967 entitled “POROUS STRUCTURE WITH METALLIC PARTICLES FOR FLUOROSCENCE SENSING” filed on Apr. 30, 2002 and U.S. provisional application No. 60/416,112 entitled “POLYMERS WITH METALLIC PARTICLES FOR USE IN FLUOROSCENCE SENSING AND FLOW SENSING APPLICATIONS” filed on Oct. 4, 2002.

### STATEMENT REGARDING FEDERALLY SPONSORED RESEARCH

The work leading to this invention was supported in part by the U.S. Government under grant number RR-08119 awarded by the NIH National Center for Research Resources. Therefore, the U.S. Government may have certain rights in this invention.

### BACKGROUND OF THE INVENTION

I. Field of Invention

The present invention relates to compositions and methods for increasing and detecting the fluorescence of fluorescent and non-fluorescent compounds, including biomolecules. The present invention also relates to methods for detecting the presence of compounds, including biomolecules.

II. Description of Related Art

Over the past 10 years fluorescence has become a dominant technology in medical testing, drug discovery, biotechnology and cellular imaging. The use of fluorescence technology has greatly enhanced the ability to detect specific molecules leading to rapid advancements in diagnostics. For example, fluorescence detection is widely used in medical testing and DNA analysis because of the high degree of sensitivity obtained using fluorescent techniques. Small numbers of molecules can be detected using fluorescence technology. Typically, extrinsic fluorophores are added covalently or non-covalently to allow molecules that do not ordinarily fluoresce or do not fluoresce at useful levels to be detected. Biomolecules, such as DNA, ordinarily do not fluoresce at detectable levels, and extrinsic fluorophores are added to DNA to facilitate the detection of DNA on gels (Benson et al. (1993) Nucleic Acids Res. 21, 5720-5726; Benson et al. (1995) Ananl. Biochem. 231, 247-255), in DNA sequencing (Smith et al. (1986) Nature 321, 674-679; Prober et al. (1987) Science 238, 336-343; Li et al. (1999) Bioconjugate Chem. 10, 241-245), in fluorescence in-situ hybridization (Denijn et al. (1992) APMIS 100, 669-681; Wiegant et la. (2000) Genome Res. 10, 861-865), and for reading of DNA arrays for gene expression (Lipshutz et al. (1999) Nat. Genet. SuppL. 1, 20-24; Ferea et al. (1999) Curr. Opin. Genet. Dev. 9, 715-722). Extrinsic fluorophores are used with DNA because DNA absorbs in the UV region near 260 nm. The short absorption wavelength is now less of an obstacle because UV solid state lasers have become available. Nonetheless, the intrinsic fluorescence from DNA is of little practical usefulness because of the low quantum yields of 10⁻⁴ to 10⁻⁵ (Vigny et al. (1974) Photochem. Photobiol. 20, 345-349; Morgan et al. (1980) Photochem. Photobiol. 31, 101-113). Because the intrinsic emission from DNA, nucleotides and nucleic acid bases is very weak (Kneipp et al. (1999) Curr. Science 77, 915-924; Nie et al. (1997) Science 275, 1102-1106; Michaels et al. (1999) J. Am. Chem. Soc. 121, 9932-9939), it is difficult to observe the intrinsic fluorescence even with modem instrumentation (Gersten et al. (1985) Surface Science 158, 165-189; Lakowicz (2001) Anal. Biochem. 298, 1-24).

Some of the fluorescence techniques used to detect the presence of molecules include Resonance Energy Transfer (RET), immunofluorescent assays, and fluorescence in situ hybridization. Detection of the molecule of interest is generally limited by the properties of the fluorophore used. In some cases, labeling a biomolecule with an extrinsic fluorophore can alter the biological activity of the biomolecule potentially creating experimental artifacts. Problems with current fluorescent techniques stem in part from the low fluorescent intensities of commonly used fluorophores. Additionally, background fluorescence can be significant when using low wavelength excitation radiation required by some fluorophores or when large quantities of fluorophore are required.

DNA sequencing techniques using fluorescent dyes as markers have their maximum emission spectra in the visible range, the DNA is subject to irradiation in the visible spectra, and visible spectra detectors and light sources are used. Generally photomultiplier tubes are used for detection. As a result, these DNA sequencing techniques have several disadvantages including high costs resulting from the high cost of the lasers used to excite the fluorescent markers which typically emit in the visible region of light spectrum and the high noise to signal ratio due to the background interferences by biomolecules.

Similarly, there has been overwhelming driving forces in analytical, biomedical and materials sciences to fabricate ever faster and smaller devices with enhanced sensitivities, precision and specificity, all with the ultimate goal of engineering devices at both the cellular and molecular level.

U.S. Appln. No. 10/073,625, which is incorporated by reference in its entirety, discloses compositions and methods for increasing fluorescence intensity of molecules, including intrinsic fluorophores and extrinsic fluorophores, which are added to allow molecules that do not ordinarily fluoresce or do not fluoresce at previously commercially useful levels to be detected. U.S. Appln. No. 10/073,625 discloses metal particles and biomolecules positioned at a distance apart sufficient to adjust intrinsic emission of electromagnetic radiation from the biomolecule in response to an amount of exciting electromagnetic radiation.

An object of the present invention is to use Surface Enhanced Fluorescence (SEF) (also “Radiative Decay Engineering”) in Biophotonics, to enable the fine detection with optical sensing and resolution and nano-sensor materials and techniques.

Additional objects of the present invention include:

Metallic geometries, using silver island films, colloids and silver-silica composites for use in surface enhanced fluorescence;

Using a layer or multi-layers of fatty acids deposited by the Langmuir-Blodgett technique to vary the inert spacer layer between the fluorophore and metal;

Using a layer or multi-layers of fatty acids deposited by the Langmuir-Blodgett technique to vary the colloid coating thickness;

Using a layer or multi-layers of polymer films vary the inert spacer layer between the fluorophore and metal;

Lining or embedding metal particles within porous silica;

A layer or multi-layers of polymer films vary the inert spacer layer between the fluorophore and metal;

Quantified fluorescence enhancement due to an increase in excitation rate and due to an increase in radiative decay rate;

Increased photostability for fluorophore-metal combinations as compared to free space fluorophores;

Application of the SEF effects to probes to incorporate non-fluorescent (low quantum yield) biomolecules of interest;

Nano-sensors based on the enhanced fluorescence properties of functional probes, e.g. for Cl⁻, I⁻, Ca²⁺ etc, located at predetermined geometries; and

Use of 3D fluorescence enhancing flow matrices, i.e. porous matrices and polymers with geometries that will afford the specific distance-dependence required for a substantial increase in the radiative decay rate of weakly fluorescing species and/or enhance the properties of functional probes which are sensitive to diffusing analytes.

### BRIEF SUMMMARY OF THE INVENTION

The present invention makes use of the technology whereby metallic particles can interact with fluorophores, producing ultra-bright fluorescence. The fluorophores produced are more photostable and may emit 10⁶ more photons per fluorophore before photodestruction.

An object of the present invention is a material or system comprising a metal particle and a compound capable of fluorescing, wherein the metal particle and the compound are separated by at least one film spacer layer. The thickness of said film is be chosen so as to enhance the fluorescence of said compound due to the distance of said compound from said metal particle. The film spacer layer may be one or multiple layers of a polymer film, a layer formed from a fatty acid or a layer formed from an oxide. The layer formed from a fatty acid may be formed by a Langmuir-Blodgett technique. The film spacer layer may be a spin coated polymer film. The oxide layer may be formed from a deposition technique, such as vapor deposition.

Another object of the present invention is a material comprising a metal particle and a compound capable of fluorescing, wherein the material comprises multiple metal particles in the form of a porous three dimensional matrix. The three dimensional matrix may be a nano-porous three dimensional matrix. The metal particles may comprise metal colloid particles and/or metal-silica composite particles. The metal particles may comprise agglomerated metal particles and/or binary linked particles or metal particles in a polymer matrix. The three dimensional matrix may be formed from controlled pore glasses or using matrices assembled from the aggregation of silver-silica composites themselves.

Another object of the present invention is a method for detecting the presence of a compound comprising spacing the compound at a distance from a metal particle with a film, exposing the compound and the metal particle to radiation; and detecting the fluorescent emission, wherein the distance provides an enhanced fluorescence intensity of the compound. The film and material structures may be the same as discussed above.

Another object of the present invention is a method for detecting the presence a compound comprising flowing said compound through a porous three dimensional matrix comprising multiple metal particles, exposing the compound and the metal particles to radiation and detecting a fluorescent emission, wherein the metal particles provide an enhanced fluorescence intensity of the compound. The three dimensional matrix and material structures may be the same as discussed above. Further, the three dimensional matrix may have an affinity for specific molecules or may filter molecules according to size.

The matrices may be metallic nanoporous matrix, through which species will flow and be both detected and counted more efficiently. Additionally, the efficiency of single molecule counting as fluorophores flow through the matrix may be improved. The ability to quantitatively count single flowing molecules under practical conditions may have many implications for medical diagnostics, the detection of biohazard organisms and new and quicker methods for DNA sequencing.

In a preferable embodiment, the film spacer layers and the metal particle coating are chemically inert and do not bind to the compounds to be detected or to intermediates that are bound to the compounds to be detected, for example covalently bound. That is, they are not reactive and are not biorecognative (not capable of binding the compound to be detected directly or by means of an intermediate binding molecule). This applies to the 2D as well as the 3D embodiments. In a preferable embodiment, the metal particles and the metal films are also inert and do not bind to the compounds to be detected or to intermediates that are bound to the compounds to be detected.

In a preferable embodiment, the compound capable of fluorescing remains free in solution.

The compound capable of fluorescing may be an inherent fluorophore or a compound attached to an extrinsic fluorophore.

The present invention will be described below in detail, but is not limited hereto.

### BRIEF DESCRIPTION OF THE DRAWINGS

FIG. 1 depicts the effects of metallic colloids in the proximity of a fluorophore.

FIG. 2 depicts Classical Jablonski diagrams for the free space condition and the modified form in the presence of metallic particles, islands or colloids. E—Excitation; Γₘ—radiative rate in the presence of metal.

FIG. 3 depicts Metal-induced effects on the fluorescence quantum yield (left) and lifetime (right).

FIG. 4 depicts the lifetime of Eu³⁺ ions in front of a silver mirror as a function of the separation between the Eu³⁺ ions and the mirror. The solid curve is a theoretical fit.

FIG. 5 depicts fluorescence decay of Eu³⁺.

FIGS. 6A and 6B are emission spectra of rhodamine B and Rose Bengal between silver island films in the presence and absence of quartz slides. S—silver; Q—quartz slides.

FIG. 7A are reconstructed time-domain intensity decays of Rhodamine B and Rose Bengal in cuvettes (C), between unsilvered quartz slides (Q) and between silvered quartz slides (S).

FIG. 8A depicts silver islands on quartz surfaces.

FIG. 8B depicts a sample geometry for silver islands on quartz surfaces with a biomolecule solution in a sandwich structure.

FIG. 9 depicts a graphical depiction of the enhancement of the emission of fluorophores having different quantum yields when placed between silver island films.

FIG. 10 are the emission spectra of calf thymus DNA in a cuvette, between silver island films and uncoated quartz plates.

FIG. 11 depicts cross sections of flow matrices.

FIG. 12 depicts a Langmuir-Blodgett technique for depositing the first monolayer of a fatty acid spacer film onto back to back silvered quartz slides.

FIG. 13 depicts examples of geometries of Langmuir-Blodgett spacer films.

FIG. 14 depicts 2D sensors using SEF.

FIG. 15 depicts absorption spectra of gold colloidal particles.

FIG. 16 depicts absorption cross sections for a silver sphere in water (−) and for prolate spheroids with axial ratios of 2.0 and 3.0 in the small particle limit.

FIG. 17 depicts examples of nano-sensors with inner silica cores.

FIG. 18 depicts the principle of size exclusion/exclusion enhanced fluorescence flow sensing. F is trapped and its fluorescence enhanced from within the pores. F₁ is not detected.

FIG. 19 depicts an example of a silver-silica colloidal building block.

FIG. 20 depicts 3D silica-silver enhancing matrix.

FIG. 21 depicts enhanced fluorescence flow sensing in a packed column.

FIG. 22 depicts protein binding and enhanced fluorescence flow sensing in a packed column.

FIG. 23 depicts enhanced fluorescence flow sensing and detection or imaging of single molecules.

FIG. 24 depicts the use of a porous lens.

FIG. 25 depicts the structure of a porous lens.

FIG. 26 depicts a porous matrix with a corrugated surface for directed emission.

FIG. 27 depicts the silanol condensation to form a binary composite.

FIG. 28 depicts the use of enhanced and directed emission of DNA using intrinsic base fluorescence.

FIG. 29 depicts fluorescence image of FITC-HSA deposited on a silver fractal-like structure and the emission spectra of the numbered areas.

FIG. 30 depicts a TEM image of SiO₂ coated Ag colloids (not aggregated).

### DETAILED DESCRIPTION OF THE INVENTION

Most of the knowledge about fluorescence is based on measurements of the spectroscopic properties of fluorophores that upon excitation, radiate into a homogeneous and non-conducting medium, typically referred to as free space. These spectral properties are well described by Maxwell's equations for a radiating oscillating dipole. However, the interactions of an emitting dipole with physical objects can be considerably more complex, as known from antenna and receiver design. The size and shape of an antenna are designed with the goal of directing the radiation and accounting for its interactions with the earth's surface.

A fluorophore is like an antenna, which oscillates at high frequency and radiates short wavelengths. Local effects are not usually observed because of the small size of fluorophores relative to the experimental apparatus.

However, literature is rapidly emerging whereby nearby conducting metallic surfaces can respond to a fluorophore oscillating dipole and modify the rate of emission, that is the intrinsic radiative decay rate, and the spatial distribution of the emitted radiation. Theoreticians describe this effect as due to changes in the photonic mode density near the fluorophore.

In most spectroscopic measurements, the solutions or medium is transparent to the emitted and sampling radiation. However, there are several important exceptions to the free space condition.

One well-known example is Surface Enhanced Raman Scattering (SERS). It is known that the presence of a metallic surface can enhance the Raman signals by factors of 10³ to 10⁸ and reports of even larger enhancements have appeared. The presence of a nearby metal film, island or particle can also alter the emission properties of fluorophores.

The most well-known effect is the quenching of fluorescence by a nearby metal. The emission of fluorophores within 50 Å of a metal surface is almost completely quenched. This effect has been used in fluorescence microscopy with evanescent wave excitation. The emission from membranes cellular regions near the quartz-water interface is quenched, allowing selective observation of the emission from the cytoplasmic region more distance from the solid-liquid interface.

In addition to quenching, it is known that metal surfaces or particles can cause increases in fluorescence. Remarkably, depending on the distance and geometry, metal surfaces or particles can result in enhancement factors of up to 1000 for the fluorescence emission.

Fluorophores near a metal surface are not expected to emit isotropically, but rather the emission is directed into selected directions, depending on the sample configuration and the nature of the metallic surface. In addition to directionality, the decay time of the fluorophores may be altered by the metal. In fact, the lifetimes of the fluorophores placed at fixed distances from a continuous metallic surface oscillate with distance.

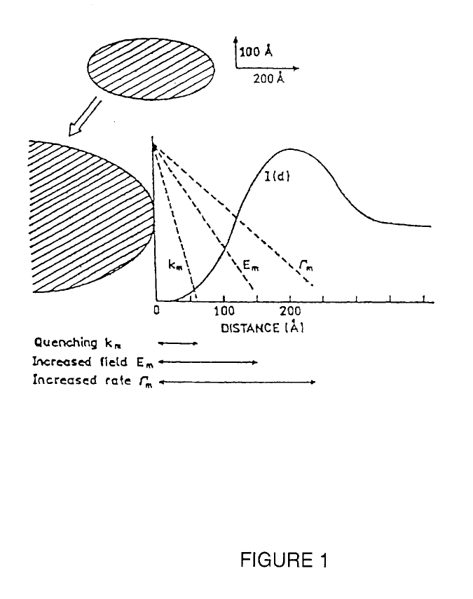

The effects of metallic particles and surface on fluorophores are due to at least three known mechanisms as described in FIG. 1. The actual distances shown in FIG. 1 are estimated and exemplary.

The first mechanism is energy transfer quenching, kₘ, to the metals with a d-⁻³ dependence. This quenching can be understood by damping of the dipole oscillations by the nearby metal.

The second mechanism is an increase in the emission intensity due to the metal increasing the local incident field on the fluorophore, Eₘ, with a maximum theoretical enhancement effect of 140. This effect has been observed for metal colloids and is appropriately called the “Lightening Rod effect.” This enhancement can be understood as due to the metal particles on concentrating the local field and subsequently increasing the rate of excitation.

The third mechanism is that a nearby metal can increase the intrinsic decay rate of a fluorophore, Γₘ, that is, to modify the rate at which the fluorophore emits photons.

It is the last and lesser-known fluorophore-metal interaction which the present inventors consider offering a remarkable opportunity for advancing fluorescence technology and is a focus of the present invention.

A few publications to date concerning SEF, for the most part, have been theoretical predictions. No experimental investigation of these effects has been reported, using well-defined sample geometries, i.e. quantitative experiments in which the fluorophore-surface distance, the size and shape of the particles and islands as well as the particle coating thickness are controlled.

Increase in Radiative Decay Rate

In fluorescence, the spectral observables are governed by the magnitude of Γ, the radiative rate, relative to the sum of the non-radiative decay rates, kₙᵣ such as internal conversion and quenching. In the absence of metallic particles or surfaces then the quantum yield, Qₒ and fluorescence lifetime τₒ are given by:

$$\begin{matrix}{Q}_{0} = \frac{Γ}{Γ + {k}_{nr}} & ( 1 ) \\ {τ}_{0} = \frac{1}{Γ + {k}_{nr}} & ( 2 )\end{matrix}$$

Fluorophores with high radiative rates have high quantum yields and short lifetimes. Increasing the quantum yield requires decreasing the non-radiative rates kₙᵣ, which is often only accomplished when using a low solution temperature or a fluorophore bound in a more rigid environment. The natural lifetime of a fluorophore, τₙ, is the inverse of the radiative decay rate or the lifetime which would be observed if their quantum yields were unity. This value is determined by the oscillator strength (extinction coefficient) of the electronic transition. The extinction coefficients of chromophores are only very slightly dependent on their environment. Hence, for almost all examples currently employed in fluorescence spectroscopy, the radiative decay rate is essentially constant.

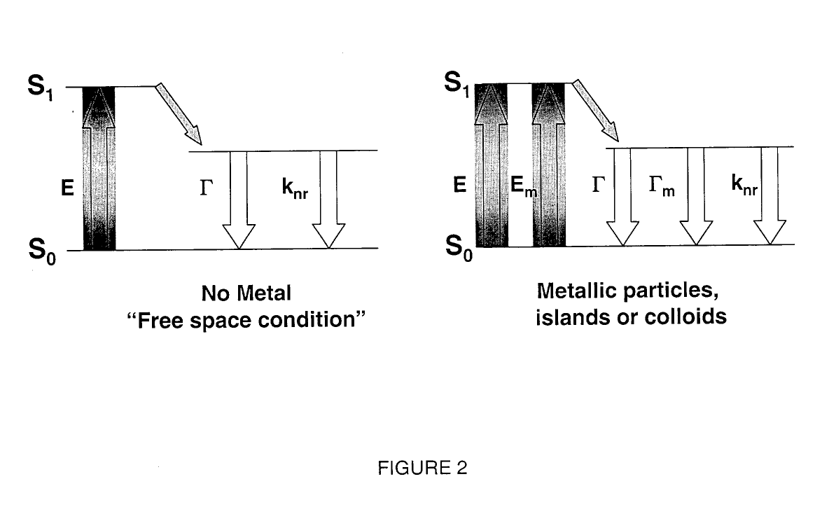

The concept of modifying the radiative decay rate of fluorophores is unfamiliar to most spectroscopists. It is intuitive to consider the novel effects of fluorescence enhancement due to metal particles, m, by assuming an additional radiative rate, Γₘ (FIG. 2). In this case, the quantum yield and lifetime are given by:

$$\begin{matrix}{Q}_{m} = \frac{Γ + {Γ}_{m}}{Γ + {Γ}_{m} + {k}_{nr}} & ( 3 ) \\ {τ}_{m} = \frac{1}{Γ + {Γ}_{m} + {k}_{nr}} & ( 4 )\end{matrix}$$

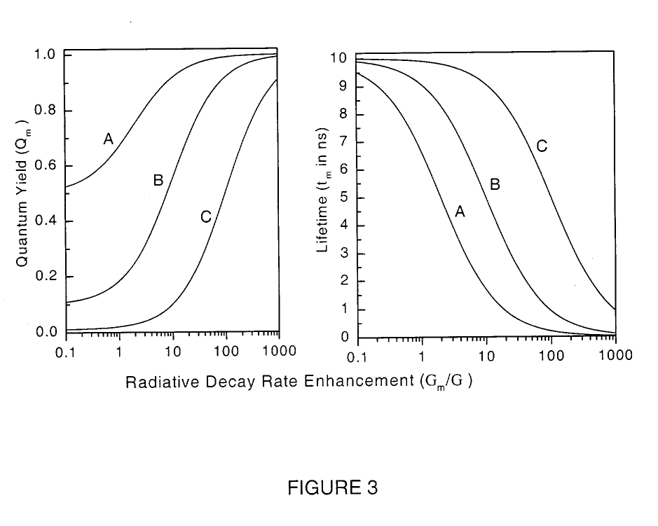

Based on these equations, it is predicted that for a fluorophore near a metal surface, as Γₘ increases, the fluorescence quantum yield increases while the lifetime decreases (FIG. 3), which is converse to the free space condition where both change in unison. An ability to modify and control the radiative decay rate (Γ+Γₘ) can have profound implications for the use of fluorescence in basic research and its applications. The modification and control of the radiative rate have also been referred as Radiative Decay Engineering (RDE), or “lightening rod” fluorescence enhancement effect.

The plots in FIG. 3 have been calculated using equation (3), assuming three fluorophores with a good 0.5 (A), low 0.1 (B) and very low quantum yield 0.01 (C), with a lifetime of 10 ns. The largest enhancement in quantum yield was observed for weak fluorophores. Accordingly, it can be predicted that fluorophores with quantum yields of about 0.001 (practically non-fluorescing) may become highly fluorescent (quantum yield˜1.0) near a metal surface, with a maximum enhancement factor of 1/Qₒ.

For example, enhanced intrinsic DNA fluorescence above metallic particles have recently been observed, which is typically not readily observable because of DNA's very low quantum yield of less than 10⁻⁴. The second favorable “lightening rod” effect also increases the fluorescence intensity by locally enhanced excitation. In this case, emission of fluorophores can be substantially enhanced irrespective of their quantum yields.

The reduction in lifetime of a fluorophore near a metal is due to an interaction between the fluorophore and metal particle, which enhances the radiative decay rate (quantum yield increase) or depending on distance, d⁻³, causes quenching. FIG. 3 (right) shows the effect of modifying the radiative decay rate on the fluorescence lifetime. For the calculations, Equation (4) was used, assuming a free space lifetime of 10 ns.

It should be noted that lifetimes of fluorophores with high quantum yields (0.5) would decrease substantially more than the lifetimes of those with low quantum yields (0.1 and 0.01). A shorter excited-state lifetime also allows less photochemical reactions, which subsequently results in an increased fluorophore photostability.

Fluorophore photostability is a primary concern in many applications of fluorescence. This is particularly true in single molecule spectroscopy. A shorter lifetime also allows for a larger photon flux. The maximum number of photons that are emitted each second by a fluorophore is roughly limited by the lifetime of its excited state.

For example, a 10 ns lifetime can yield about 10⁸ photons per second per molecule, but in practice, only 10³ photons can be readily observed. The small number of observed photons is typically due to both photo-destruction and isotropic emission. If a metal surface decreases the lifetime, one can obtain more photons per second per molecule by appropriately increasing the incident intensity.

On the other hand, the SEF effects provide enhanced intensity, while simultaneously shorten the lifetime. That is, it may be possible to decrease the excitation intensity, yet still see a significant increase in the emission intensity and photostability. Such unique concepts in fluorescence will likely be useful in novel biotechnologies as well as applications in imaging.

The ability to increase the radiative decay rate suggests that any chromophore, even non-fluorescent species such as bilirubin, fullerenes, metal-ligand complexes or porphyrins could display usefully high quantum yields when appropriately placed near a metal surface. The effects of metal surface-fluorophore interactions are highly dependent upon the distance between the metal surface and the species, and the nature of the metal surface.

The emission enhancement may be observed at distances according to the type of fluorophore to be detected and the type of metal. For example, emission enhancement may be observed when a fluorophore distances about 5 nm to about 200 nm to metal surfaces, c.f. FIG. 1. Preferable distances are about 5 nm to about 30 nm, and more preferably, 5 nm to about 20 nm to metal surfaces. At this scale, there are few phenomena that provide opportunities for new levels of sensing, manipulation, and control. In addition, devices at this scale may lead to dramatically enhanced performance, sensitivity, and reliability with dramatically decreased size, weight, and therefore cost.

Different effects are expected for mirrors, sub-wavelength or semi-transparent metal surfaces, silver island films or metal colloids. There have been reports on surface enhanced fluorescence, using metal islands, metal colloids, metal surfaces or mirrors. More dramatic effects are typically observed for islands and colloids as compared to continuous metallic surfaces.

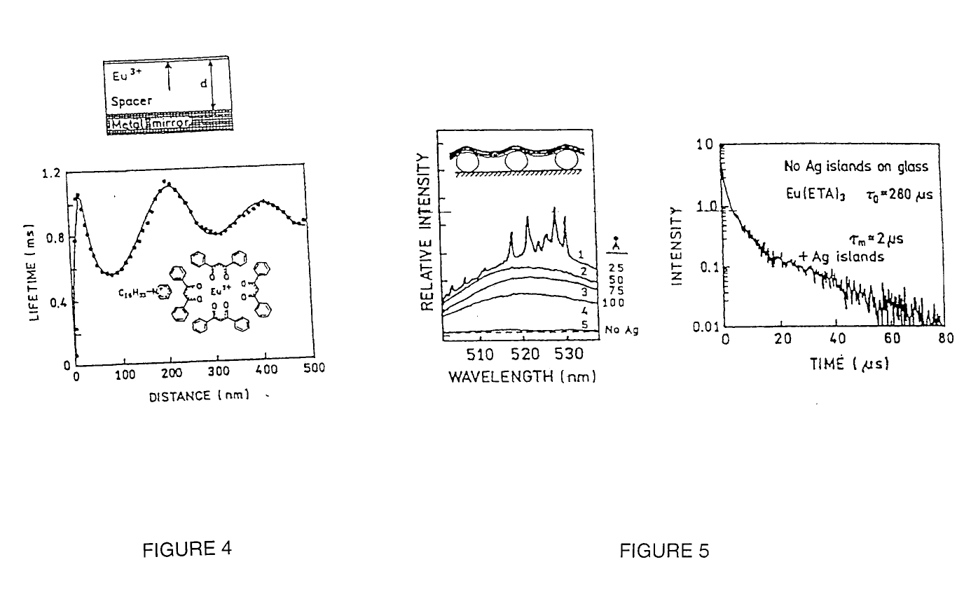

The possibility for altering the radiative decay rate was demonstrated by measurements of the decay times of europium (Eu³⁺) positioned at various distances from a planar silver mirror using Langmuir-Blodgett films. In a mirror, the metal layer is continuous and thicker than that for a semi-transparent film. The lifetimes of Eu³⁺ oscillate with distance from the metal, yet still remain a single exponential at each distance (FIG. 4). The oscillating lifetime can be explained by changes in the phase of the reflected field with distance and the effects of the reflected field on the fluorophore.

Specifically, a decrease in lifetime is found when the reflected field is in phase with the fluorophore. As the distance increases, the amplitude of the oscillations decreases. At short distances, for example, below 20 nm, the emission is quenched, c.f. FIG. 1. This effect is due to a coupling of the fluorophore dipole to the surface plasmon resonance of the metal, oscillating surface charges on the metals surface. The lifetimes typically oscillate at around 25% the free space value. However, more dramatic effects are observed with small metal particles (FIG. 5 left).

Similarly, the effects of silver islands were also examined, wherein the silver islands were coated with a thin film of Eu(ETA)₃, where ETA is a ligand that chelates europium. When the Eu³⁺ chelate was deposited on a silica substrate without the silver islands, it displayed a single exponential decay time of 280 μs and a quantum yield near 0.4 (Qₒ is typically 0.4). However, when deposited on silver island films, the intensity increases about 5-fold (not shown) and the lifetime decreased by ca. 100 fold to near 2 μs (FIG. 5).

Further, the decay is no longer a single exponential on the silver island films. The silver islands had the remarkable effect of increasing the intensity 5-fold while decreasing the lifetime 100-fold. Such an effect can only be explained by an increase in the radiative decay rate, c.f. equations 3 and 4.

In this sample geometry, an inert coating between the islands prevents Eu³⁺ chelates between the islands from being emissive. The 5-fold increase in the quantum yield of Eu(ETA)₃ results in an apparent quantum yield of 2.0, which by definition is impossible. Hence, this additional enhancement must be due to an increase in the local excitation field near the metal particles.

It should be noted that this increase in local intensity of the incident light cannot explain the decreased lifetime, because an unperturbed Eu³⁺ chelate excited by this enhanced field would still decay with a 280 μs lifetime, i.e. enhanced excitation results in a visual increase in fluorescence and does not alter the fluorescence lifetime. Interestingly, these large increases in the radiative decay rate are due to the fluorophores near the metallic particles.

In most cases, it is generally observed that dramatic increases occur for metallic surfaces. For example, Raman signals are dramatically enhanced by metal colloids or islands.

Fluorescence can be detected using devices including, but not limited to, a spectrofluorometer having a light source and detector. Light sources can include arc lamps and lasers. Detectors can include photomultiplier tubes. Additionally, it is advantageous for the device to have a monochromator so that specific wavelengths of light may be used to excite a molecule or to detect emissions at a specific wavelength. When a sample containing a fluorophore is placed in the spectrofluorometer and exposed to an amount of exciting radiation, the fluorophore emits radiation that is detected by a photomultiplier tube. The fluorescence intensity of a biomolecule can be increased in response to an amount of exciting radiation when the distance between the metal particle and the biomolecule is from about 50 Å to about 2000 Å, preferably from about 50 Å to about 200 Å. Alternatively, the fluorescence intensity of the biomolecule can be reduced when the distance between the biomolecule and the metal particle is less than about 50 Å.

Another embodiment provides a method for manipulating fluorescence intensity of a biomolecule including the steps of increasing the rate of radiative decay of the biomolecule by positioning the biomolecule at a distance from a metal particle, and exposing the biomolecule to an amount of exciting radiation. By increasing the rate of radiative decay, the fluorescence intensity of the biomolecule can be increased. It has been discovered that by manipulating the distance separating a biomolecule and a metal particle, the radiative decay of the biomolecule can also be manipulated.

In another embodiment, the present invention provides a method for identifying nucleic acids, the method including the steps of positioning a nucleic acid a distance from a metal particle, irradiating the nucleic acid, detecting the fluorescence emission from the nucleic acid, and identifying the nucleic acid based on the fluorescence emission. The identification of a nucleic acid using the intrinsic fluorescence of the nucleic acid eliminates the requirement for extrinsic probes. In one embodiment, the background fluorescence is not problematic because the intrinsic fluorescence can be increased by about 80 fold thereby reducing the noise to signal ratio. In another embodiment, the nucleic acid can be identified based on the emission spectra obtained from monitoring the fluorescence of the sample. Thus, the sequence of nucleic acids in a sample can be determined by sequentially removing a nucleic acid, positioning the nucleic acid adjacent to metal particle, irradiating the nucleic acid with an amount of exciting radiation, detecting the emitted radiation, and correlating the emitted radiation with the nucleic acid base. Methods for sequentially removing a single nucleic acid form a nucleic acid sequence such as an oligonucleotide are known in the art and include sequential digestion, hydrolysis, and chemical cleavage. The nucleic acids can be positioned a distance from a metal particle by causing the stream of a fluid sample containing a nucleic acid to pass near a surface containing the metal particle. The metal particles of such surfaces can be thin films or islands of metal that form part of a sample chamber. The irradiation of the nucleic acid can be timed to coincide with the positioning of the nucleic acid adjacent to the metal. The nucleic acids can be irradiated with one or more wavelengths. In a preferred embodiment, the nucleic acids are excited at wavelengths below 300 nm, preferably from 280 to about 295 nm. In another embodiment, the excitation wavelength is near 520 nm for multi-photon excitation.

Still another embodiment provides a method for increasing the fluorescence intensity of a fluorescently labeled biomolecule including the steps of labeling a biomolecule with a fluorophore, positioning the labeled biomolecule adjacent to a metallic particle such that in response to an amount of exciting radiation, the fluorophore emits radiation, preferably detectable amounts of radiation. In a preferred embodiment, the fluorophore has a quantum yield of less than 0.8, preferably less than 0.5, more preferably less than 0.2, and most preferably less than 0.1. In this embodiment, the fluorescence intensity of an extrinsic fluorophore can be used to detect the biomolecule.

The present invention provides a method for increasing the intrinsic fluorescence of a biomolecule including the step of positioning a metal particle and the biomolecule at a distance apart sufficient to increase the electromagnetic emission from the biomolecule in response to an amount of exciting radiation. It will be appreciated that the present invention includes positioning of a biomolecule adjacent to a metal particle or positioning a metal particle adjacent to biomolecule in any of the disclosed embodiments.

The present invention provides a method for detecting a biomolecule including the steps of positioning a metal particle and a biomolecule at a distance apart sufficient to manipulate the electromagnetic emission from the biomolecule, exposing the biomolecule to an amount of exciting radiation, and detecting the electromagnetic emission from the biomolecule.

The present invention provides a method for increasing the fluorescence intensity of a fluorescently labeled biomolecule including the steps of labeling a biomolecule with a fluorophore, positioning the labeled biomolecule at a distance apart from a metallic particle such that in response to an amount of exciting radiation, the fluorophore emits radiation.

The term “fluorophore” means any substance that emits electromagnetic energy such as light at a certain wavelength (emission wavelength) when the substance is illuminated by radiation of a different wavelength (excitation wavelength). Extrinsic fluorophores refer to fluorophores bound to another substance. Intrinsic fluorophores refer to substances that are fluorophores themselves. Exemplary fluorophores include but are not limited to those listed in the Molecular Probes Catalogue which is incorporated by reference herein. Representative fluorophores include but are not limited to Alexa Fluor® 350, Dansyl Chloride (DNS-Cl), 5-(iodoacetamida)fluoroscein (5-IAF); fluoroscein 5-isothiocyanate (FITC), tetramethylrhodamine 5-(and 6-)isothiocyanate (TRITC), 6-acryloyl-2-dimethylaminonaphthalene (acrylodan), 7-nitrobenzo-2-oxa-1,3,-diazol-4-yl chloride (NBD-Cl), ethidium bromide, Lucifer Yellow, 5-carboxyrhodamine 6G hydrochloride, Lissamine rhodamine B sulfonyl chloride, Texas Red™ sulfonyl chloride, BODIPY™, naphthalamine sulfonic acids including but not limited to 1-anilinonaphthalene-8-sulfonic acid (ANS) and 6-(p-toluidinyl)naphthalene-2-sulfonic acid (TNS), Anthroyl fatty acid, DPH, Parinaric acid, TMA-DPH, Fluorenyl fatty acid, Fluorescein-phosphatidylethanolamine, Texas red-phosphatidylethanolamine, Pyrenyl-phophatidylcholine, Fluorenyl-phosphotidylcholine, Merocyanine 540, 1-(3-sulfonatopropyl)-4-[β-[2 [(di-n-butylamino)-6 naphthyl]vinyl]pyridinium betaine (Naphtyl Styryl), 3,3′dipropylthiadicarbocyanine (diS-C₃-(5)), 4-(p-dipentyl aminostyryl)-1-methylpyridinium (di-5-ASP), Cy-3 lodo Acetamide, Cy-5-N-Hydroxysuccinimide, Cy-7-Isothiocyanate, rhodamine 800, IR-125, Thiazole Orange, Azure B, Nile Blue, Al Phthalocyanine, Oxaxine 1, 4′, 6-diamidino-2-phenylindole (DAPI), Hoechst 33342, TOTO, Acridine Orange, Ethidium Homodimer, N(ethoxycarbonylmethyl)-6-methoxyquinolinium (MQAE), Fura-2, Calcium Green, Carboxy SNARF-6, BAPTA, coumarin, phytofluors, Coronene, and metal-ligand complexes. Representative intrinsic fluorophores include but are not limited to organic compounds having aromatic ring structures including but not limited to NADH, FAD, tyrosine, tryptophan, purines, pyrirmidines, lipids, fatty acids, nucleic acids, nucleotides, nucleosides, amino acids, proteins, peptides, DNA, RNA, sugars, and vitamins. Additional suitable fluorophores include enzyme-cofactors; lanthanide, green fluorescent protein, yellow fluorescent protein, red fluorescent protein, or mutants and derivates thereof.

The term “biomolecule” means any carbon based molecule occurring in nature or a derivative of such a molecule. The biomolecule can be in active or inactive form. “Active form” means the biomolecule is in a form that can perform a biological function. “Inactive form” means the biomolecule must be processed either naturally or synthetically before the biomolecule can perform a biological function. Exemplary biomolecules include nucleic acids, aromatic carbon ring structures, NADH, FAD, amino acids, carbohydrates, steroids, flavins, proteins, DNA, RNA, oligonucleotides, peptide nucleic acids, fatty acids, sugar groups such as glucose etc., vitamins, cofactors, purines, pyrimidines, formycin, lipids, phytochrome, phytofluor, peptides, lipids, antibodies and phycobiliproptein.

A dramatic increase in fluorescence emission of Cardio Green (indocyannine green), which is widely used in retinal angiography, can be observed when it is coated both between and on, silver colloids coated on a substrate (U.S. provisional application No. 60/409,851, incorporated by reference herein in its entirety).

The metal particles used in the present invention can be spheroid, ellipsoid, or of any other geometry. The metal particles can be suspended in a colloid or combination of colloids, alloys, or combinations of more than one metal. The metal particles can be placed on substrate surfaces as thin films, or deposited on surfaces to form small islands. The surfaces can be metallic or non-metallic. Additionally, the metal particles can be coated with polymers, gels, adhesives, oxides, SiO₂, or biologic material. Exemplary coatings include substances that increase the binding of the metal particle to surfaces or other molecules. The metal particles may be layer(s) of metal formed or coated on non-metal particles. Metal particles, preferably noble metals, most preferably silver, may be chemically reduced on a surface. Exemplary substrate surfaces include but are not limited to glass or quartz.

Exemplary metals include, but are not limited to, rhenium, ruthenium, rhodium, palladium, silver, copper, osmium, iridium, platinum, and gold. Metal particles or metal films are known and can be produced using known methods. U.S. application Ser. No. 10/073,625, which is incorporated by reference in its entirety, discloses examples of preparing metal particles and metal films.

Preparation of Metal Islands

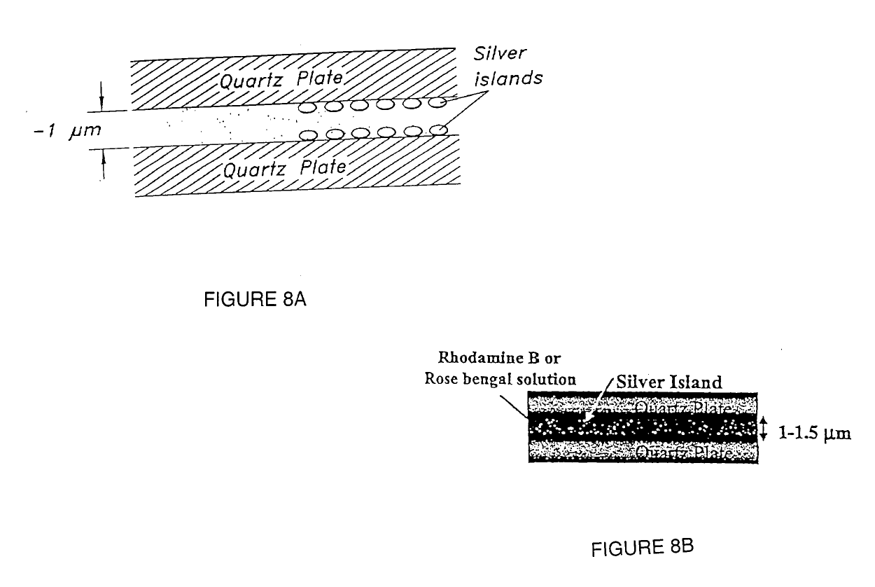

The metal surfaces for SEF can be obtained using metal island films, sandwiched films (FIG. 8A) or even spin coated silver islands or colloids. A quartz surface is preferred for forming the metal islands. Prior to use, the quartz slides are soaked in 10 parts 98% H₂SO₄ and 1 part 30% H₂O₂ for at least 24 hours.

The island particles are prepared in clean beakers by reduction of metal ions using various reducing agents. Rivas L., Sanchez-Cortes S., Garcia-Ramos J. V. and Morcillo G., Growth of Silver Colloidal Particles Obtained by Citrate Reduction to Increase the Ramen Enhancement Factor, Langmuir, 17(3), 574-577 (2001). For example, sodium hydroxide is added to a rapidly stirred silver nitrate solution forming a brown precipitate. Ammonium hydroxide is added to re-dissolve the precipitate. The solution is cooled and dried quartz slides are added to the beaker, followed by glucose. After stirring for 2 minutes, the mixture is warmed to 30° C. After 10-15 minutes, the mixture turns yellow-green and becomes cloudy. A thin film of silver particles has formed on the slides as can be seen from their brown green color. The slides are rinsed with pure water prior to use.

Alternative procedures for preparing metal particles are also available. Shirtcliffe N., Nickel U. and Schneider S., Reproducible preparation of silver sols with small particle size using borohydride reduction: For use as nuclei for preparation of large particles, J. Colloid Interface Sci., 211(1), 122-129 (1999); Pastoriza-Santos I., and Liz-Marzan L. M., Reduction of silver nanoparticles in DMF. Formation of monolayers and stable colloids, Pure Appl. Chem., 72(1-2), 83-90 (2000); Pastoriza-Santos I., Serra-Rodriquez C. and Liz-Marzan L. M., Self-assembly of silver particle monolayers on glass from Ag⁺ solutions in DMF, J. Colloid Interface Sci., 221(2), 236-241 (2000); Bright R. Musick M. D. and Natan M. J., Preparation and characterization of Ag colloid monolayers, Langmuir, 14(20), 5695-5701 (1998); Ni F. and Cotton T. M., Chemical procedure for preparing surface-enhanced Raman scattering active silver films, Anal. Chem., 58(14), 3159-5163 (1986). Silver is primarily used because of the familiar color from the longer surface plasmon absorption of silver.

It is also possible to silanize the slides by placing them in a 2% solution (v/v) of 3-aminopropyltrimethoxysilane in dry methanol for 2 hours, rinsing and then air-drying. The silanized substrates should be used within one hour or stored under a dry nitrogen atmosphere.

It is necessary to use fresh metal surfaces and to avoid oxidation or other chemical reactions on the surface. The surface plasmon absorption can be used to monitor the quality of the metal islands.

(b) Preparation of Silver Colloids

Colloids can be prepared as suspensions by citrate reduction metals. Preferred metals are silver and gold. Again, gold may be avoided because of the absorption of gold at shorter wavelengths. However, gold colloids may be used with longer wavelength red and NIR fluorophores.

The size of the colloids and their homogeneity can be determined by the extensive publications on the optical properties of metal particles available and the effects of interface chemistry on the optical property of colloids. Krelbig U., Gartz M. and Hilger A, Mie resonances: Sensors for physical and chemical cluster interface properties, Ber. Bunsenges, Phys. Chem., 101(11), 1593-1604. (1997).

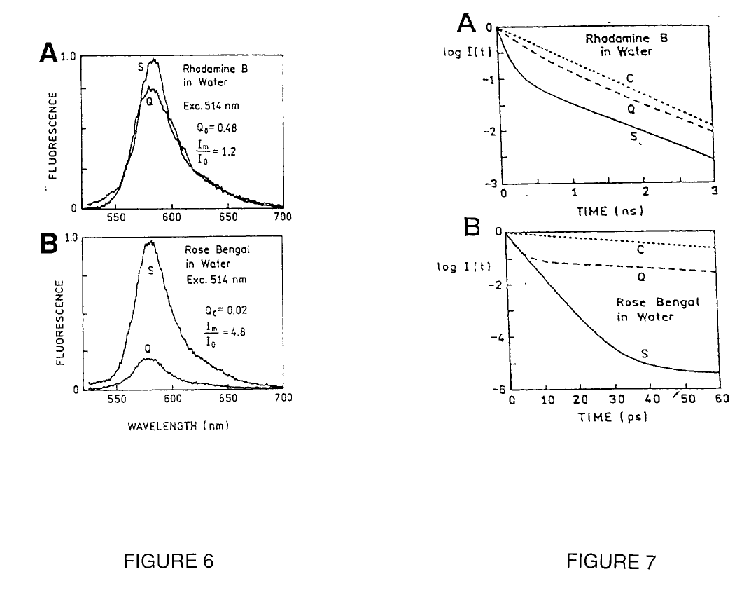

Inventors first investigated surface enhanced fluorescence by using low (Rose Bengal Qₒ=0.02) and high quantum yield fluorophores (Rhodamine B (RhB) Qₒ=0.48), and silver island metal films (FIGS. 6 and 7). Silver island films are made by depositing silver on a glass substrate and consist of sub-wavelength size silver particles. Under appropriate conditions, the glass becomes covered with circular islands about 200 Å in diameter. Typically, about 40% of the surface is covered by silver.

FIG. 8B shows an example of the use of the two plate geometry and flowing the solution containing the compound to be detected therebetween. For RhB, the intensities are nearly equivalent between unsilvered quartz plates (Q) and the silver island films (S). The small enhancement of RhB (FIG. 6) is expected because for high quantum yield fluorophores, the radiative rate cannot be substantially increased, where the quenching interaction with the metal and the excitation enhancement effects are likely to compete.

If there is an excitation enhancement effect in this sample, it is believed to be offset by the quenching effect, kₘ. In any event, the emission intensity increased for Rose Bengal (the lower quantum yield species) and this excitation enhancement is present during the excitation of both RhB and Rose Bengal. For Rose Bengal, it was observed a remarkable 5-fold increase in intensity (FIG. 6B). This increase is especially remarkable because only a small fraction of the volume between the films is within this enhancing region.

This interaction region extends until about 200 Å into the solution. Given a 1 μm thickness and the presence of two films, only about 4% of the sample can be within this enhancement region. This exciting result suggests a very high quantum yield for Rose Bengal molecules adjacent to the surface, in fact greater than unity, which can only be explained by a complimentary increase in the rate of excitation also.

Lifetime measurements are also informative (FIG. 7), as the intensity measurements shown in FIG. 6 might under normal circumstances be explained by an increased rate of excitation or enhanced fluorescence due to the fluorophore bound to the surface of the quartz. However, lifetime measurements are unambiguous which showed a substantial reduction in lifetime for Rose Bengal between silvered quartz plates and remained roughly constant for RhB. The slight drop in RhB lifetime can be explained by some RhB molecules being within 50 Å of the silver and hence, being quenched. The bi-exponential decays can be explained in terms of sample heterogeneity.

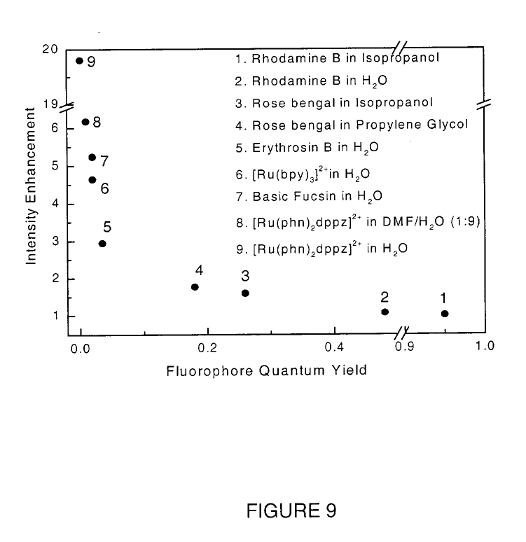

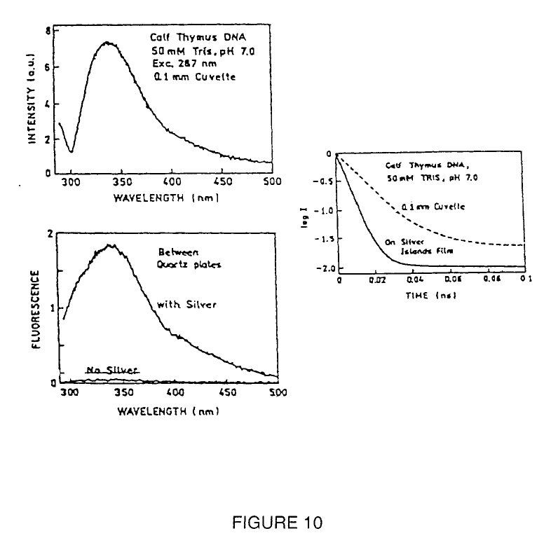

The Inventors subsequently examined several other fluorophores of different well-characterized free space quantum yields (FIG. 9). In all cases, it was found the largest enhancements with the lowest quantum yield fluorophores, confirming the predictions of enhanced fluorescence due to both an increase in radiative decay rate and enhanced excitation. Amazingly, the intrinsic fluorescence of double helical DNA can be enhanced using the same sample geometry. For example, calf thymus DNA showed a significant enhancement when placed between the silver island coated slides (FIG. 10).

Silver island films used in these experiments were formed by a chemical reduction of a silver salt on the quartz surface, which are relatively simple to fabricate. However, this approach does not provide a control of particle size, or distance of the fluorophores from the surface. As shown in FIG. 8B, much of the sample is indeed distant from the metal islands.

Enhancements of 1000 fold have been with the realization that sample geometries have been heterogeneous and the enhancement factors spatially averaged. Fluorescein-labelled Human serum albumin was coated onto a surface where silver fractal like structures had been grown. The Inventors reasoned that these structures would provide for multiple 3D opportunities for fluorophore-metal interactions. Indeed, images of the fluorescein coated fractal-like structures revealed fluorescent hot spots with some localized enhancement factors of many thousand fold FIG. 29. Geddes, Chris D., Parfenov, Alexandr, Roll, David, Gryczynski, Ignacy, Malicka, Joanna and Lakowicz, Joseph R., Roughened Silver Electrodes for use in Metal-Enhanced Fluorescence, and Parfenov, Alexandr, Gryczynski, Ignacy, Malicka, Joanna, Geddes, Chris D., and Lakowicz, Joseph R., Enhanced Fluorescence from Fluorophores on Fractal Silver Surfaces, both are incorporated by reference herein in their entirety. Intuitively, these fluorescent hot spots are thought due to specific fluorescein molecules which are at the appropriate distance for optimum fluorophore-metal interactions. Applicants believe that the unique distance dependences for optimum enhancement can be predetermined on a planar surface, and can then be adapted into a 3D porous structure. A 3D porous structure is likely to provide for many more opportunities for fluorophore-metal interactions as compared to fractal structures on a planer surface.

More quantitative measurements may be obtained by using samples in which the fluorophores are at known distances from the metal. These samples may be prepared in several manners as will be described below. However, the present invention is not limited to those described herein.

Metal particles can be bound to a surface by placing functional chemical groups such as cyanide (CN), amine (NH₂) or thiol (SH), on a glass or polymer substrate. Metal colloids are known to spontaneously bind to such surfaces with high affinity. Freeman R. G., Grabar K. C., Allison K. J., Bright R. M., Davis J. A., Guthrie A. P., Hommer M. B., Jackson M. A., Smith P. C., Walter D. G. and Natan M. J., Self-assembled metal colloid monolayers: An approach to SERS substrates, Science, 267, 1629-1632 (1995); Grabar K. C., Freeman R. G., Hommer M. B. and Natan M. J., Preparation and characterization of Au colloid monolayers, Anal. Chem., 67, 735-743 (1995).

Positioning of the biomolecule or metal particle at a desired distance can be achieved by using a film. The film may be a polymer film, a Langmuir-Blodgett film or an oxide film.

(1) Langmuir-Blodgett Films

Metal-fluorophore distances may be achieved by using Langmuir-Blodgett films with fatty acid spacers. The fatty acids may be from natural sources, including concentrated cuts or fractionations, or synthetic alkyl carboxylic acids. Examples of the fatty acids include, but not limited to, caprylic (C₈), capric (C₁₀), lauric (C₁₂), myristic (C₁₄), palmitic (C₁₆), stearic (C₁₈), oleic (C₁₈), linoleic (C₁₈), linolenic (C₁₈), ricinoleic (C₁₈) arachidic (C₂₀), gadolic (C₂₀), behenic (C₂₂) and erucic (C₂₂). The fatty acids with even numbered carbon chain lengths are given as illustrative though the odd numbered fatty acids can also be used.

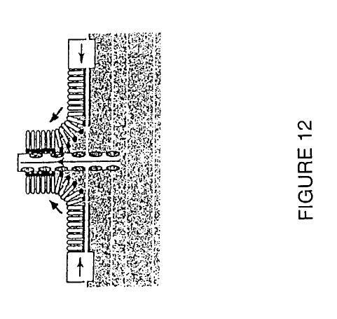

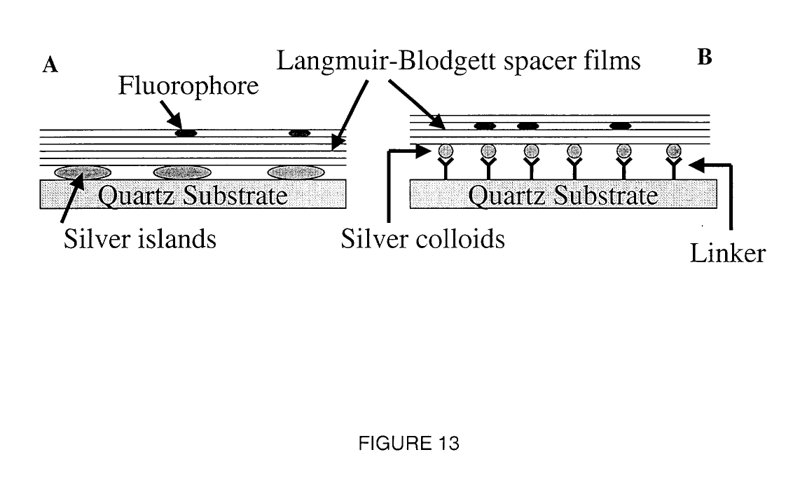

The Langmuir-Blodgett technique provides an accurate means of controlling film thickness and surface uniformity, and was originally used to obtain the data for Eu³⁺ shown in FIG. 4. This technique allows an accurate control of the metal-fluorophore distance. A commercially available device (for example, KSV 5000 III “Alternative Layer Dipping Trough”) which allows a one to lay down different numbers of fatty acid layers, with an additional final layer containing the desired fluorophore, now positioned at a suitable distance, to enable SEF may be used (FIGS. 12 and 13).

While the thickness of these monolayers is well characterized, ellipsometry can be used to further confirm the thickness of the fatty acid layers. Ellipsometry measures the phase difference in the polarization of linearly polarized light on reflection from a surface.

Since fresh silver island and colloidal films can be readily generated, and fluorophores can be positioned at known geometries, this approach permits investigation of the distance dependence of SEF. Additionally, Aroca and co-workers have done some pioneering work on surface enhanced resonance Raman Linker scattering of LB monolayers on silver island films.

The use of “known thickness” Langmuir-Blodgett films is diagrammatically shown in FIGS. 13A and 13B. Langmuir-Blodgett films can be used as inert spacer layers above silver islands or above bound silver colloids, as shown in FIGS. 13A and 13B, respectively.

(2) Polymer Films

Metal-fluorophore distances may be achieved by using polymer films. Examples of the polymer include, but not limited to, polyvinyl alcohol (PVA). Absorbance measurements and ellipsometry may be used to determine polymer film thickness.

One type of polymer films is spin coated polymer films. The technology of spin coated polymer spacer films readily allows films to be coated onto a variety of surfaces, with varied thickness from >0.1 μm. The coating can be performed on a spin coater, which allows uniform surface thickness by varying polymer concentration (viscosity) and spin speed. For example, Model P6700 spin coater (Specialty Coating Systems Inc.), allows uniform surface thickness by varying polymer concentration (viscosity) and spin speed.

(3) Oxide Films

The film spacer layer may be one or multiple layers formed from an oxide. The oxide layer may be formed from a deposition technique, such as vapor deposition. Preferably, the oxide is a silicon oxide, more preferably, SiO₂.

The vapor deposition of SiO₂ is a well established technique for the controlled deposition of a variety of substrates. An Edwards Vapor deposition module allows the deposition of silver island films of known thickness and mass, while also depositing an inert spacer layer of SiO₂ without breaking the vacuum of the system (dual trough). This minimizes any potential oxidation effects on the metal. It may be possible that vapor deposition can be used to coat the inside of pores with silver also. This would provide for an alternative method for matrix I fabrication, diminishing the need for wet silver chemistries.

In a preferable embodiment, the film spacer layers are not biorecognative layers. This applies to the 2D as well as the 3D embodiments.

Surface Plasmon Absorption Measurements

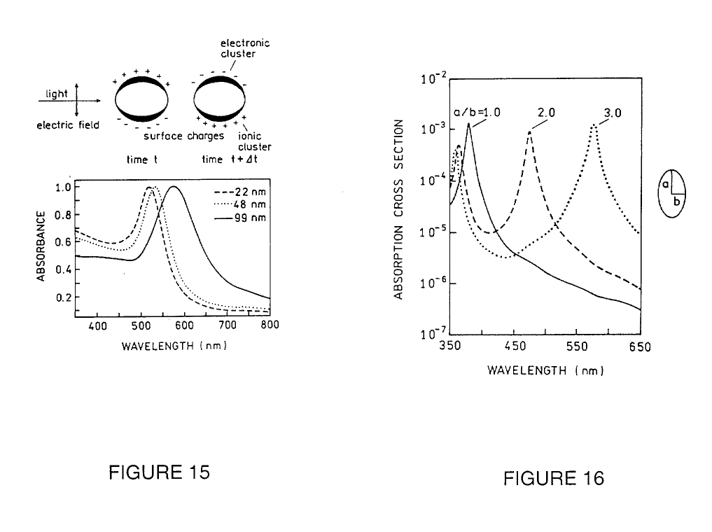

The surface plasmon absorption is due to the oscillations of free charges at a metal boundary which propagate along the metal surface. These resonance's are often excited using evanescent waves. The surface plasmon absorption can give an indication of colloid size (FIG. 15) and shape (FIG. 16).

The prepared colloid and island samples can be characterized using published optical properties of these metal particles. Link S. and El-Sayed M. A., Spectral properties and relaxation dynamics of surface plasmon electronic oscillations in gold and silver nanodots and nanorods, J. Phys. Chem. B., 103, 8410-8426 (1999); Kreibig U. and Genzel L., Optical absorption of small metallic particles, Surface Science, 156, 678-700 (1985). This effect depends on the refractive index of the medium, and under certain conditions, is sensitive to binding to the surface of the metal. While any binding effects on the surface plasmon absorption can be monitored using simple absorption measurements, functionalization of only the outer inert silica coatings typically does not change the plasmon absorption to any great extent.

To generate stable silver nano-particles with the surface free electron density as high as possible, fresh colloids may be coated with inert silica (which may later be functionalized for sensors) from sol-gel solutions using known procedures. Esumi K., Suzuki A., Yamahira A. and Torigoe K., Role of poly(amidoamine) dendrimers for preparing nanoparticles of gold, platinum and silver, Langmuir, 16, 2604-2608 (2000). In addition, other methods to protect the metal surfaces and colloids have been reported, such as by coating with polyvinylpyrrolidone (PVP) if desired.

(2) Silver-Silica (Ag-SiO₂) Composites and Nano-Flow Matrix

In addition to silver island and colloidal films, it is also possible to prepare bimetallic metal nanoparticles, Toshmia N. and Yonezawa T., Bimetallic nanoparticles-novel materials for chemical and physical applications, New J. Chem., 1179-1201 (1998), or hollow sphere colloids Caruso F., Caruso R. A. and Mohwald H, Nanoengineering of inorganic and hybrid hollow spheres by colloidal templating, Science, 282, 1111-1114 (1998). The enhancement is expected for a fluorophore positioned in the center of a hollow silver sphere, which is transparent to the fluorescence emission.

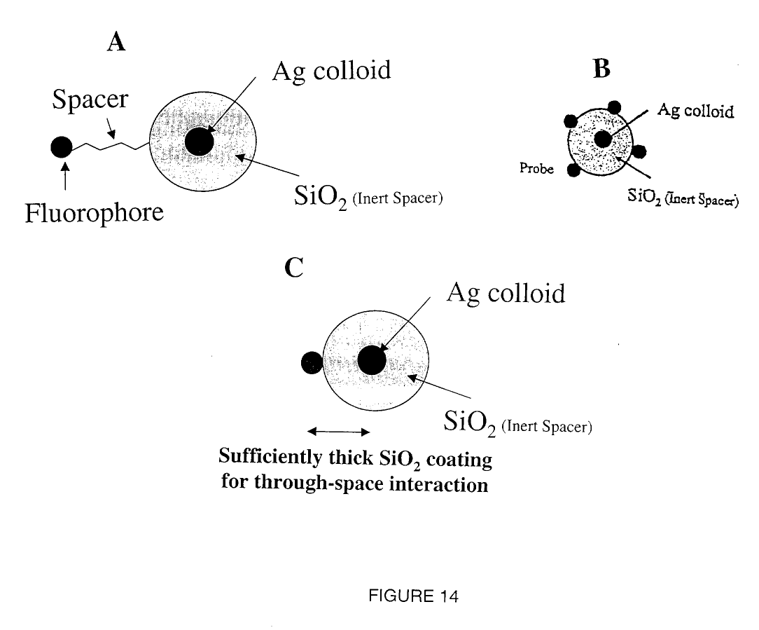

Metallic particles can also be coated with inert silica spacers, and the silica can then be derivatized by methods used for attaching organic molecules to glass. Yee J. K., Parry D. B., Caldwell K. D. and Harris J. M., Modification of quartz surfaces via thiol-disulphide interchange, Langmuir, 7, 307-313 (1991). 2D enhancing sensors may be assembled by this method (FIG. 14A). Procedures for coating particles with silica have been developed as means to alter the spectral properties of semiconductor nanoparticles. Farmer S. C. and Patten T. E., Synthesis of luminescent organic/inorganic polymer nanocomposites, Polym. Mater. Sci. Eng., 82, 237-238 (2000); Comor M. I. and Nedeljkovic J. M., Enhanced photocorrosion stability of colloidal cadmium sulphide-silica nanocomposites, J. Mater. Sci. Lett., 18, 1583-1585 (1999). The inert silica spacers can function to provide the spacing between the metal and the compound capable of fluorescing.

For 2D nano-sensors, silica colloids and islands may be initially prepared. The specific size to be used may be determined based on the results obtained in the experiments using Rose Bengal and rhodamine B.

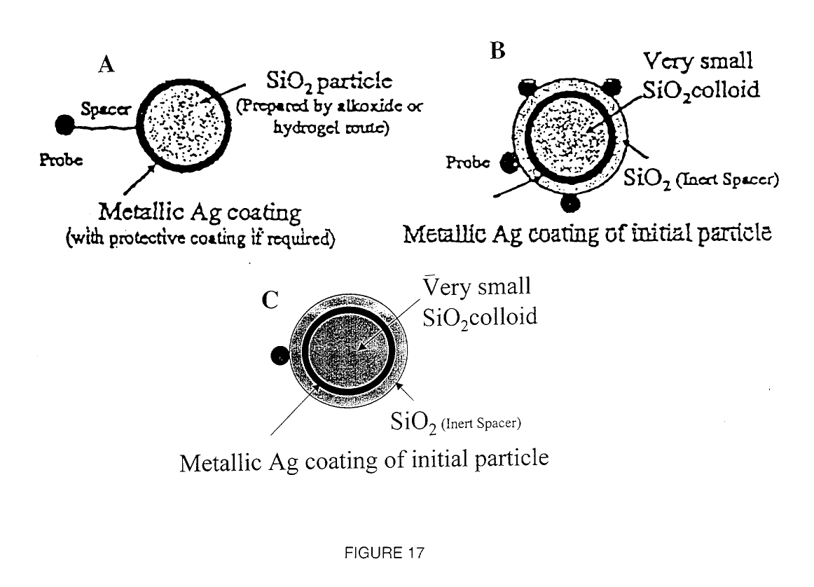

In these experiments, the distance dependencies may be quantified. Subsequently, functional probes may be positioned at the specific geometries. The present inventors have previously shown that one can readily control the size and distribution of silica nano-particles produced via the sol-gel route. Geddes C. D. and Birch D. J. S., Nanometre resolution of silica hydrogel formation using time-resolved fluorescence anisotropy, J. Non-Cryst. Sol., 270(1-3), 191-204 (2000); Birch D. J. S. and Geddes C. D., Sol-gel particle growth studied using fluorescence anisotropy: An alternative to scattering techniques, Phys. Rev. E., 62(2), 2977-2980 (2000); Karolin J., Geddes C. D., Wynne K., and Birch D. J. S., Nanoparticle metrology in sol-gels using multiphoton excited fluorescence anisotropy decay, Meas. Sci. Technol., 13, 21-27 (2002); Geddes C. D., Karolin J. and Birch D. J. S., 1 and 2-photon fluorescence anisotropy decay in silicon alkoxide sol-gels: Interpretation in terms of self-assembled nanoparticles, Jn. Phys. Chem. B.—In Press (2002); Birch D. J. S. and Geddes C. D., Cluster Dynamics, Growth and Syneresis during Silica Hydrogel Polymerization, Chem. Phys. Letts, 320(3-4), 229-236 (2000). Colloids having central silica atoms and protected outer metallic skins (FIG. 17) will enable a greater choice of nano-sensors.

One particular advantage of this approach is that extremely small silica inner colloids (<1 nm) can be produced, which when coated with a metal will typically produce smaller colloids than those obtained by the citrate reduction of the metal, providing more choice of colloids.

Functional Fluorescent Probes for Sensing

To fabricate SEF based nano-sensors, it is essential to determine the maximum fluorescence enhancement geometries (distance dependence) for functional fluorescent probes, weakly intrinsically fluorescent biologically important species (such as nucleotides, bilirubin, infectious agents etc), and biomolecules labeled with fluorophores.

Present inventors have previously conducted calculations for several probes. Similar calculations may also be done for many other commercially available probes, such as probes for pH, metal ion, including but not limited to Ca²⁺, Mg²⁺, Na⁺ and K⁺, and anionic ions, including but not limited to Cl⁻ and I⁻, which typically display low quantum yields (<0.1). The spectral properties of these probes may be greatly enhanced using SEF, by characterizing them near/above silver islands at specific distances. Two discreet enhancing matrices are envisaged. One is the inclusion/exclusion sensing of weakly intrinsically fluorescent species, such as nucleotides, bilirubin etc. The second is to use the matrix to enhance the properties of functional probes which are themselves sensitive to diffusing analytes, such as Cl⁻ and Ca²⁺ etc.

By controlling the probe environment, such as modifying the pH or Ca²⁺ concentration, the functional properties of the fabricated nano-sensors in terms of enhanced fluorescence and improved photostability may be determined. After each environmental change, spectroscopic data may be acquired, analyzed and assessed in terms of the probe functionality in various nano-sites. Such measurements will allow immediate comparison of probes that display substantial enhancement due to the appropriate proximity to the metal surface and those which are not affected (i.e. too far from metal surface) and can be used for fluorescence sensors on the nanometer scale.

However, it is likely that the analytical properties of probes in the presence of metal surfaces may be different than those in bulk solution. This is because many probes change their spectroscopic properties when bound to specific analyte and subsequently, SEF effects may be different between those observed for the free form of the probe and those of analyte-bound one.

The greatly enhanced fluorescence of the nano-sensors in this embodiment of the present invention would overcome many difficulties associated with background fluorescence from biological specimens, i.e. significantly increase the signal to noise ratio.

3D Silica-Silver Enhancing Matrices

Further, by aggregating the nano-colloids shown in FIG. 14B, a porous matrix may be produced. By using the geometries shown in FIG. 14A, a second porous matrix may be produced, which will enhance the spectral properties of functional probes that are entrapped and sensitive to diffusing analytes. The size (and distribution) of the pores in the matrices can be readily controlled by changing the aggregating solution conditions, such as pH and temperature. Brinker C. J. and Scherer G., Sol-Gel Science, The Physics and Chemistry of Sol-Gel processing (Academic press, San Diego, 1989); Iler R. K., in The Chemistry of Silica, (Wiley, New York 1979).

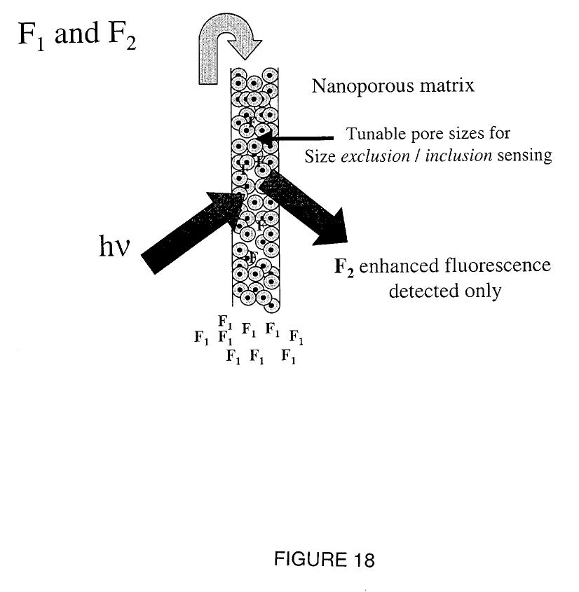

This technique for controlling silica morphology is widely used to manufacture many forms of silica, such as chromatography silica, fining silica gels, and etc. Iler R. K., in The Chemistry of Silica, (Wiley, New York 1979). It is also possible that by controlling the pore size distribution of the matrix, which is indirectly controlled by the size of the aggregating SiO₂-Ag composites, to fabricate novel size inclusion/exclusion fluorescence enhancing silica matrices (FIG. 18). Such a material may have applications in high sensitivity sensing, such as DNA sequencing.

A 3D structure having silica-coated metal colloids may be assembled into a 3D porous network, wherein weakly fluorescing species, such as nucleotides or even bilirubin, are positioned automatically at the maximum enhancing geometry. Another 3D structure may provide a matrix to enhance the properties of functional probes which are themselves sensitive to diffusing analytes.

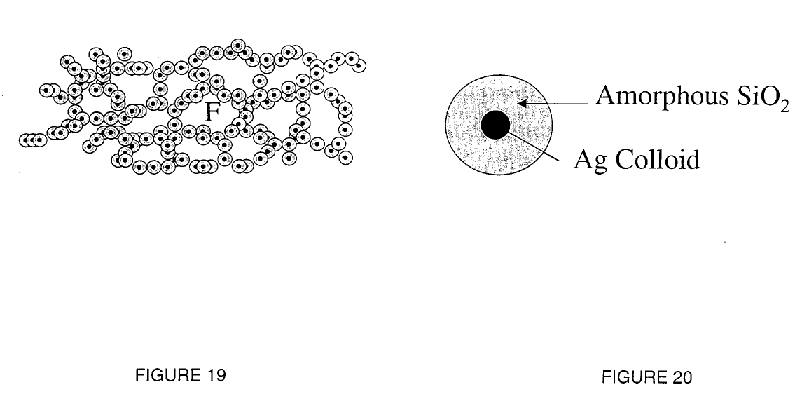

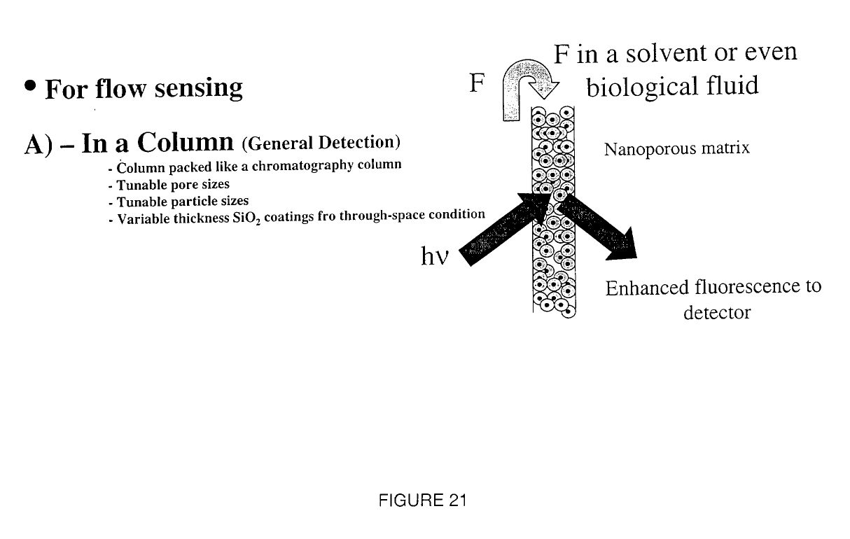

A sensing matrix may be self-assembled via the sol-gel route using metal coated metal colloids. FIG. 19 exemplifies an example of a silver-silica colloidal building block. The self-assembled sol-gel type porous silica-silver matrix with tunable pore and volume sizes may provide a substantial increase in the radiative decay rates of weakly fluorescent species encapsulated with the 3D structure (FIG. 21). The 3D structure may be in any form, including a packed column (FIG. 21) or a monolith.

A porous silica matrix may also be used for size exclusion or inclusion sensing, based upon the pore sizes of the silica to detect flowing species through the metallised porous silica.

Porous glasses with known pore size distributions, may be purchased from a variety of U.S. companies including Geltech and Corning (Corning's Vycor glasses) (Controlled pore glasses, CPLs). Metal island films may be deposited on the inside of the pores. As an alternative to using known porous glasses which will be lined with silver island films, a porous glass may be created, where the silver particles are embedded within the walls of the glass. This can be achieved by aggregating SiO₂ coated silver colloids together at a known pH and particle density.

Inorganic or organic glasses may be used.

Matrix 1: Controlled pore glasses (lined silver). Controlled pore glasses are typically densified glasses with a known surface area and pore volume/size. The densification of these porous structures can render them relatively inert to a variety of pH's and solvents. Glasses can be readily purchased with narrow pore size distributions as small as a few angstroms to hundreds of nanometers. In addition, glasses are readily available that have been prefunctionalized for applications such as the chemical affinity of biomolecules and have even been used as catalytic agents and bioreactors. The inside of the controlled pore glasses may be lined with silver using the procedure recently described by Selvan and co-workers. By immersing a preformed sol-gel into a AgNO₃ solution containing a reducing agent, silver island films were readily deposited on the inside of the porous structure, which was corroborated by TEM images. Interestingly, they were able to see the development of the silver island films by the appearance of the Surface Plasmon Absorption at 395 nm. Selvan et at has reported that this procedure was most effective at enhancing the fluorescence of encapsulated Eu³⁺ ions. Monitoring of the surface plasmon absorption of the silver will also give an indication of the size and shape of the silver island films as well as extent of silver deposition.

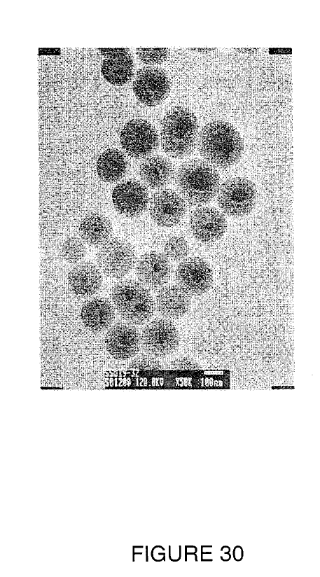

Matrix 2: The aggregation of SiO₂ coated silver colloids (Embedded silver). By control of the preparation conditions such as pH and concentration, SiO₂ coated silver nanosized particles can readily be produced with a variety of SiO₂ shell thicknesses as shown in FIG. 30. Matijexie and Hardikar report procedures for producing both aggregated and single (non-aggregated) particles. It is believed that these procedures can be adopted here, where the thickness of the SiO₂ can be tuned to correspond to the optimum distance for metal enhanced fluorescence. The aggregation of these particles will produce a porous material, where the pore size is dependent on the size, pH and the number density of the particles.

The fluorescent enhancement of the porous matrices in both static and flow modes may be tested against a control sample, which has identical geometries but without the inclusion of lined or embedded silver to determine optimum matrix fabrication.

When colloidal particles are used, the pore size distribution may be controlled by the means of controlling pH and colloid particle size, ortho/silicate concentrations or temperature, a new class of “nano type” sensing matrices may be realized.

The sensing matrixes of the present invention may have many novel features over conventional sensing matrices, such as:

Tunable radiative decay rates of the non-fluorescent species by spatially locating them in pores of tunable dimensions/volume;

The enhancement of the radiative decay rates of analytes which are usually considered to be non-fluorescent without the need for a sensing element, i.e., transduction element;

Size-exclusion/inclusion sensing of non-fluorescent species;

Single-pore single molecule detection;

The increased photostability of embedded fluorophores; a matrix which may be used for either small or large sample volumes;

A castable, coatable and drawable matrix;

An inert matrix with low thermal expansion and low UV optical transmission;

A potential completely non-leaching matrix of unbound fluorophores; and

A material which may produce porous optical components for maximum detection efficiency.

For example, in flow-type sensing, a packed column may be produced by packing a matrix of metal particles or metal colloidal particles in a column in an analogous manner to a silica column used in chromatography. The species/analyte of interest could flow through the structure and be subsequently detected by its fluorescence signal/lifetime due to an increase in fluorescence quantum yield and a decrease in excited state lifetime (FIG. 21).

In size exclusion/inclusion sensing, specifically selected species/analytes can be sensed by controlling the pore size. That is, the species which are too large to diffuse with the individual pores may be readily washed out (F), while those retained within the individual pores may be detected by an increase in radiative decay rate (f) (FIG. 18) The pore size may be tunable via particle size, such as varying the thickness of the silver-silica colloid building block, for example by varying the SiO₂ thickness, for controlling the size inclusion/exclusion through space conditions. As exemplified in FIG. 18, the molecules F are too large to go through the pores and are trapped and detected and the molecules F₁ flow though and are not detected.

In one embodiment, detection occurs without binding the molecules to the sensor or support. The molecule to be detected is not chemically bound. The molecule to be detected may remain in solution and not directly or indirectly interact with the metal particles, coatings or film spacer layers.

Weakly or non-fluorescent species/analytes may be encapsulated in a matrix for single molecule studies. This enhancing matrix could potentially be very useful because the signal level in single molecule detection is often limited by the photo-decomposition of the fluorophores. Increasing the radiative decay rate of a fluorophore encapsulated within the matrix will invariably increase the quantum yield of fluorescence but reduce the lifetime, lengthening the time before photo-decomposition.

It is well known that the sol-gel route is par excellence a method for producing optical components. It may be possible to construct porous optical components based on the enhancing matrix, for further focusing and detection of low fluorescence intensities.

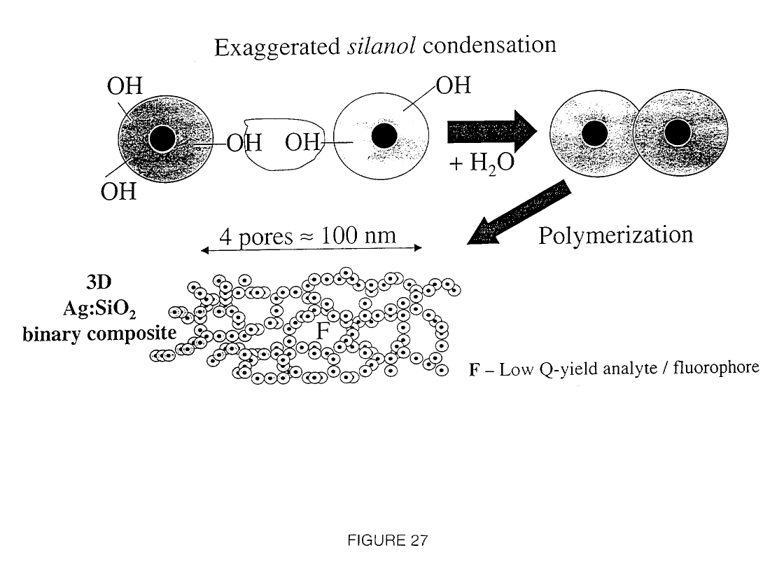

The 3D structure may also be prepared by fixing the metal colloidal particles together. The metal colloidal particles may be condensed into binary components that are used to form a composite for assembling into a 3D porous network. For example, the metal colloidal particles may be condensed via a silanol condensation reaction and use to form a binary composite that can provide a 3D structure with pore sizes of about 100 nm (FIG. 27).

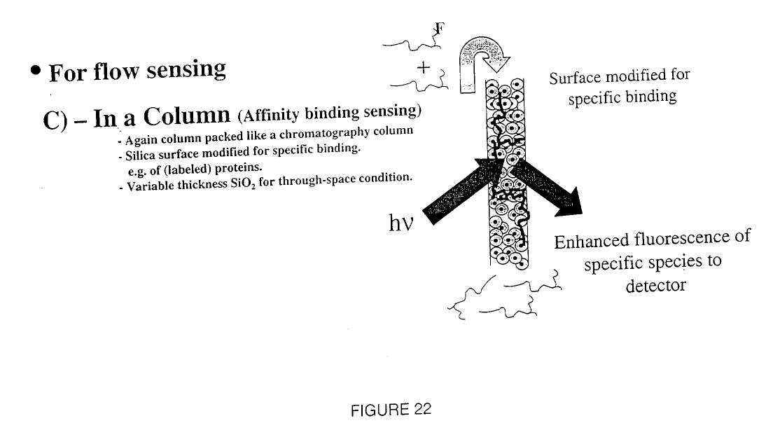

The 3D structure may also be prepared with metal colloidal particles that have been modified to provide separation based on the particular characteristics of the molecules other than size. For example, the metal colloidal particles may be surface modified for specific binding, such as for particular proteins or for labeled proteins. See FIG. 22. Variable thicknesses may also be used in combination.

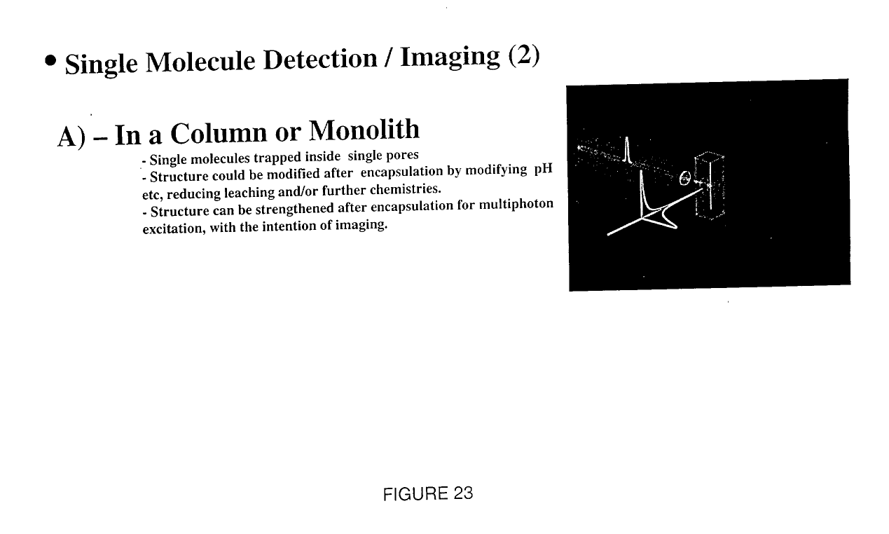

The 3D structure may be used for detection or imaging of single molecules, which are trapped inside single pores or on specific binding sites. See FIG. 23. The molecules may also be modified after encapsulation to enhance photon excitation.

Solvent-fluorophore combinations may be tested to determine which will prevent the fluorophores or labeled biomolecules from binding to the matrix, in an analogous manner to the choice of parameters for column chromatography. Testing of solvent-fluorophore combinations thus may be used to determine an optimized matrix. Availability of both silver lined and embedded matrices also provide for a greater choice of fluorophore-solvent combinations.

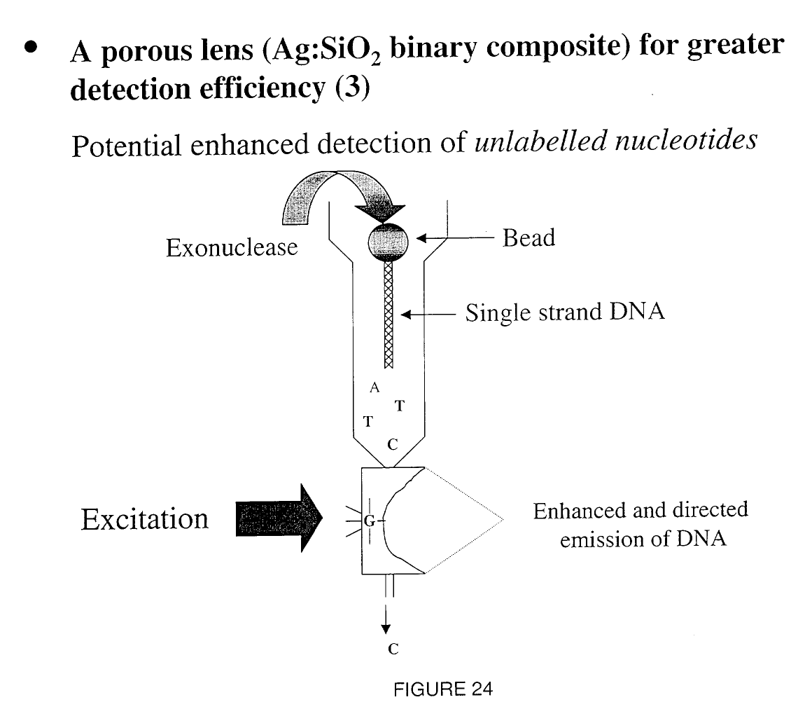

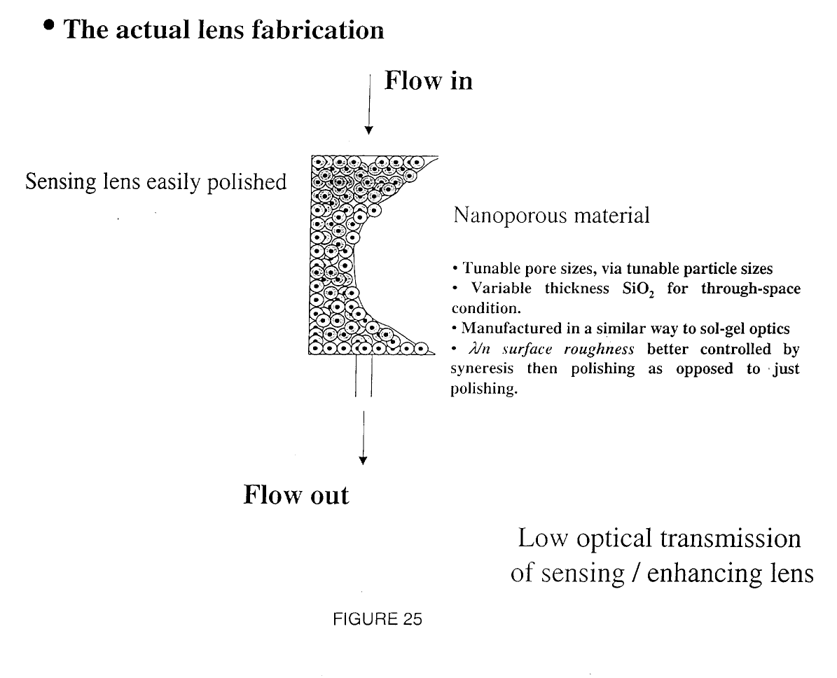

The metal colloidal particles may also be located in a shaped structure in order to enhance and direct the emission by photon excitation. For example, the shaped structure may be a lens structure of nano-porous material formed from metal colloidal particles. Such structures may be manufactured in a similar way as sol-gel optics. Such structures may be used to enhance detection of unlabelled nucleotides, DNA or RNA. See FIG. 24 and FIG. 25. The thicknesses and other separation characteristics may be used to provide low optical transmission in a sensing/enhancing lens.

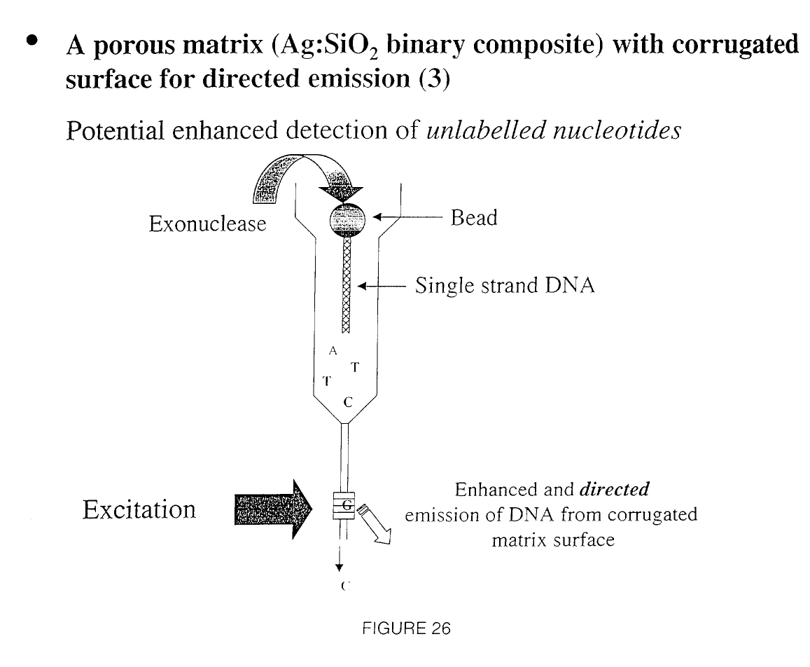

The 3D porous matrix may also be used with a corrugated surface to enhance and direct the photon excitation (FIG. 26).

Metallic colloids (or various other non-spherical shapes/particles) may also be incorporated into organic polymers, covalently or non-covalently, to form polymeric matrices, wherein the distance from diffusing species affords an increase in radiative decay rate and thus, an increase in quantum yield. Such polymeric matrices are ideal for sensing/flowing sensing applications of low concentration species.

Due to the relatively low cost, polymers are important as visible wavelength range optical components. The polymers may also provide stability to the metal particles. Metal particles Such plastic optical components may be used for sensing/flow sensing.

Polymers containing metal particles may have other applications, including but not limited to, size inclusion/exclusion sensing of non-fluorescent species, increased photostability of embedded fluorophores, single pore single molecule detection, and porous polymers which allow diffusing analytes or antibodies, resulting in a detectable and quantifiable signal change in the analyte or antibody or respective transduction element.

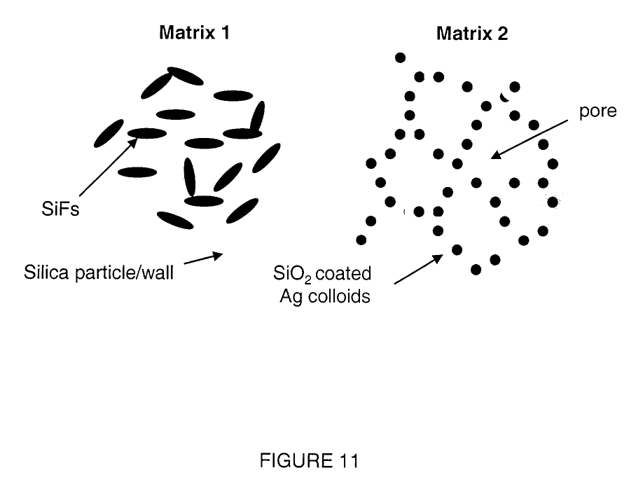

The metallic-silica nanoporous matrix may improve the efficiency of single molecule counting as fluorophores flow through the matrix. Two types of flow matrix may be used (FIG. 11). Matrix type 1 is fabricated using commercially available known pore-size glasses (Controlled pore glasses) that are to be lined with silver, while Matrix type 2 is fabricated by aggregating silica coated silver colloids together to form a 3D porous structure where the silver is embedded. While type 1, is simpler to fabricate, type 2, offers an attractive inert coating on the surface of the silver which may protect against the binding of species during flow. Research to date indicates that the slow oxide formation and the coating of SiO₂ to the surface of silver particles does not disrupt the surface plasmon resonances and therefore, the metal-enhanced fluorescence effect. Further, type 2 matrices, may be a more tunable way of controlling the through space requirements of metal-enhanced fluorescence.

This embodiment of the present invention may also have vast applications in clinical medicine, environmental monitoring applications, homeland security such as rapid detection of low concentration species, industrial processes, pharmaceutical industries such as monitoring species, and sensors for use in reduced atmospheres such as biohazard clean rooms and space light.

Multiphoton Excitation

Multi-photon excitation is now readily available, and is typically used in fluorescence microscopy and spectroscopy. Because the “lightening rod” effect effectively enhances the excitation rate, enhanced rates of multiphoton excitation is expected.

Since double-photon excitation is proportional to the square of the intensity, and the maximum enhancement due to the lightening rod effect is theoretically believed to be at 140, increases of up to 10⁴ are envisaged. Both single and double-photon excitation can be used to quantify the enhancement distances, as double-photon excitation will effectively allow a greater dynamic range of enhancement signal.

Photostability measurements

Based on the belief that the substantial reduction in fluorescence lifetime near metallic surfaces will afford an increase in probe photostability, Lakowicz J. R., Gryczynski I, Shen Y. B., Malicka J., and Gryczynski, Z., Intensified fluorescence, Photonics Spectra, 35(10), 96-104 (2001), an increase in the maximum number of photons from a single fluorophore before photobleaching is expected. Additionally, the maximum rate of photon emission per second should also increase because of the decreased lifetime. A technique, based on fluorescence correlation spectroscopy (ECS) may be used for determining these parameters. Also, Harms et al describes in detail a relatively simple technique to measure the maximum emission rate, the photobleach time and the quantum yield. Harms G. S., Cognet L., Lommerse P. H. M., Blab G. A. and Schmidt T., Autofluorescent proteins in single-molecule research: Applications to live cell-imaging microscopy, Biophys. J., 80, 2396-2408 (2001).

The benefits of the present invention include an increase in fluorescence intensity due to increases in the excitation and radiative decay rates, and the present invention has many profound implications and applications in biochemical, biophysical, clinical testing and sensing.

For example, that emission of low quantum yield chromophores can be increased has important implications for studies of nucleic acids and protein fluorescence. That emission can be made directional rather than isotropic can provide improved detectability of weak signals, with potential applications in rapid detection systems for bioterrorism-related pathogens. Likelihood that surface enhanced fluorescence can result in a million-fold more photons per fluorophore may provide an equivalent, if not surpassing PCR and ELISA in terms of sensitivity, for detection of infectious organisms without the need for the currently used amplification steps.

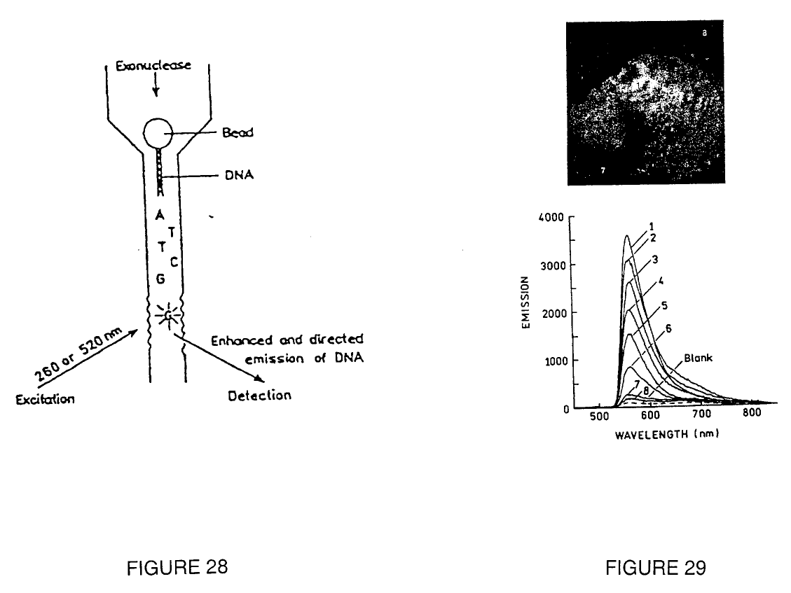

Another application of surface enhanced fluorescence could be to increase the detection of single molecules with minimal missed molecules. For example, there has been a continuing effort to sequence a single strand of DNA by the action of an exonuclease, followed by labeling, detecting and identifying each nucleotide as it is realized. Sauer M., Angerer B., Ankenbauer W., Foldes-Papp Z., Gobel F., Han K.-T, Rigler R., Schulz A., Wolfrum J. and Zander C., Single molecule DNA sequencing in submicrometer channels: state of the art and future prospects, J. Biotechnology, 86, 181-201 (2001); Enderlein J., Robbins D. L., Ambrose W. P. and Keller R. A., Molecular shot noise, burst size distribution, and single molecule detection in fluid flow: Effects of multiple occupancy, J. Phys. Chem. A., 102, 6089-6094 (1988). The difficulties currently encountered in this process lie in the detection and identification of each nucleotide as it is removed because a 100% yield for the labeling reaction is required. The present invention offers a simplified alternative by using intrinsic nucleotide fluorescence, rather than the labeled nucleotides (FIG. 28).

The use of an appropriate metal surface with known and quantified configuration could increase the quantum yield and direct the emission in the desired direction. Hence, a detailed understanding of metal enhanced fluorescence and its geometrical dependencies could facilitate single strand DNA sequencing.

Further, it has been assumed that the Raman enhancement factor of several hundreds shown in FIG. 5, is the largest which could occur. Kummerlen, J., et al, Enhanced dye fluorescence over silver island films: Analysis of distance dependence, Molecular Phys., 80(5) 1031-1046 (1993). However, recent studies of surface enhanced Raman scattering have shown that the enhancements were due to a small percentage of the silver colloid particles. These so-called “hot-particles” displayed SERS increases of 10¹⁴ to 10¹⁸, much larger than that measured for the bulk samples. Nie S. and Emory S. R., Probing single molecules and single nanoparticles by surface-enhanced Raman scattering, Science, 275, 1102-1106 (1997); Emory S. R. and Nile S., Screening and enrichment of metal nanoparticles with novel optical properties, J. Phys. Chem. B., 102, 493-497 (1998).

Applicants believe that hot particles would also be found with respect to Eₘ and Γₘ, i.e. SEF. Clusters of particles will be expected to display larger effects than isolated particles, as suggested for the large Raman enhancements.

In addition to the above-mentioned fields of potential applications of the present invention, there is a widespread interest in single molecule detection and single molecule spectroscopy. Van Holde K. E., Biochemistry at the single molecule level: Minireview series, J. Biol. Chem., 274(21), 14515 (1999); Erdmann R., Enderlein J. and Seidel C., (Eds.), Single molecule detection and ultrasensitive analysis in the life sciences, Cytometry, 36(3), 161-264 (1999); Berezhkovskii A. M., Szabo A. and Weiss G. H., Theory of the fluorescence of single molecules undergoing multistate conformational dynamics, J. Phys. Chem. B., 104, 3776-3780 (2000); Molski A., Hofkens J., Gensch T., Boens N. and DeSchryver F., A single-molecule fluorescence spectroscopy, Chem. Phys. Letts., 318, 325-332 (2000). Currently, single molecule detection is being applied to study single molecule DNA and RNA mechanisms, Bustamante C., Smith S. B., Liphardt J. and Smith D., Single molecule studies of DNA mechanics, Curr. Opin, Struct. Biol., 10, 279-285 (2002); Zhaung X., Bartley L. E., Babcock H. P., Russell R., Ha D. and Chu S.; A single-molecule study of RNA catalysis and folding, Science, 288, 2048-2051 (2000), rotational motion, Ha T., Glass J., Enderie Th., Chemla D. S. and Weiss S., Hindered rotational diffusion and rotational jumps of single molecules, Phys. Rev. Lett., 80(10), 2093-2096 (1998), and enzyme catalysis, Lu H. P., Xun L. and Xia X. S., Single-molecule enzymatic dynamics, Science, 282, 1877-1887 (1998). The present invention may aid improved detectability, in terms of increased photostability and directed emission, by modification of the fluorescence lifetimes as described herein.

U.S. Application Ser. No. 10/073,625 is incorporated by reference herein in its entirety. U.S. provisional application No. 60/376,967, U.S. provisional application No. 60/416,112 and U.S. provisional application No. 60/409,851 are incorporated by reference herein in their entirety.

Although the invention has been described with respect to specific embodiments, the details are not to be construed as limitations, for it will become apparent that various embodiments, changes and modifications may be resorted to without departing from the spirit and scope thereof, and it is understood that such equivalent embodiments are intended to be included within the scope of this invention.

What is claimed is:

1. An apparatus for detecting or measuring the presence of a compound capable of fluorescing in a sample comprising a metal in the form of a particle or a film, at least one film spacer layer, said compound capable of fluorescing, and a source of irradiation, wherein the metal particle or metal film and the compound are separated by said at least one film spacer layer.

2. The apparatus according to claim 1, wherein the metal comprises silver or gold.

3. The apparatus according to claim 1, wherein the metal comprises silver.

4. The apparatus according to claim 1, wherein the metal is in the form of a metal film.

5. The apparatus according to claim 1, wherein the apparatus comprises multiple metal particles in the form of islands on a substrate.

6. The apparatus according to claim 1, wherein the apparatus comprises multiple metal particles in the form of islands located between two substrates.

7. The apparatus according to claim 1, wherein the metal is in the form of a particle selected from the group consisting of a metal colloid particle, a metal-silica composite or a metal particle in a polymeric material.

8. The apparatus according to claim 1, wherein the film spacer layer comprises a polymer film.

9. The apparatus according to claim 8, wherein the film spacer layer comprises a PVA polymer film.

11. The apparatus according to claim 1, wherein the film spacer layer comprises a layer formed from a fatty acid by a Langmuir-Blodgett technique.

12. The apparatus according to claim 11, wherein said fatty acid is arachidic acid.

13. The apparatus according to claim 1, wherein the film spacer layer comprises silica.

14. The apparatus according to claim 13, wherein the metal is in the form of a particle embedded in said silica.

15. The apparatus according to claim 1, wherein said compound is an inherent fluorophore.

16. The apparatus according to claim 1, wherein said compound is attached to an extrinsic fluorophore.

17. The apparatus according to claim 1, wherein the thickness of said film spacer layer is chosen so as to enhance the fluorescence of said compound due to the distance of said compound from said metal.

18. An apparatus for detecting or measuring the presence of a compound capable of fluorescing in a sample, comprising metal particles, said compound capable of fluorescing, a porous substrate in the form of a three dimensional matrix and a source of irradiation, wherein the metal particles are on the surface of the porous substrate or the metal particles are embedded in the porous substrate.

19. The apparatus according to claim 18, wherein the porous substrate comprises porous silica or porous glass.

20. The material according to claim 18, wherein the porous substrate comprises the metal particles in a polymer matrix.

21. An apparatus for detecting or measuring the presence of a compound capable of fluorescing in a sample comprising metal particles, said compound capable of fluorescing, and a source of irradiation, wherein the metal particles form a porous three dimensional matrix.

22. The apparatus according to claim 21, wherein the metal particles comprises metal particles selected from the group consisting of metal colloid particles and metal-silica composite particles.

23. The apparatus according to claim 21, wherein the multiple metal particles comprise binary linked particles.

24. The apparatus according to claim 21, wherein the porous three dimensional matrix comprises agglomerated metal particles.

25. The apparatus according to claim 21, wherein the porous three dimensional matrix is in the form of a lens.

26. The apparatus according to claim 21, wherein the porous three dimensional matrix is in the form of a corrugated matrix surface.

27. The apparatus according to claim 21, wherein the porous three dimensional matrix filters molecules according to size.

28. A method for detecting the presence of a compound comprising:
spacing the compound at a distance from a metal particle with a film spacer layer;
exposing the compound to radiation; and
detecting the fluorescent emission,
wherein the distance provides an enhanced fluorescence intensity of the compound.

29. The method according to claim 28, wherein the compound is between two film spacer layers located on two substrates with multiple metal particles in the form of islands on the film spacer layers.

30. The method according to claim 29, wherein the film spacer layer comprises a spin coated PVA polymer film.

31. The method according to claim 29, wherein the film spacer layer comprises at least one layer formed from a fatty acid by a Langmuir-Blodgett technique.

32. The method according to claim 31, wherein said fatty acid is arachidic acid.

33. The method according to claim 29, wherein the film spacer layer comprises silica.

34. A method for detecting the presence of a compound comprising:
flowing said compound through a porous three dimensional matrix comprising multiple metal particles;
exposing the compound to radiation; and
detecting a fluorescent emission,
wherein the metal particles provide an enhanced fluorescence intensity of the compound.

35. The method according to claim 34, wherein the metal particles comprise metal particles selected from the group consisting of metal colloid particles and metal-silica composite particles.

36. The method according to claim 34, wherein the multiple metal particles comprise binary linked particles.

37. The method according to claim 34, wherein the porous three dimensional matrix comprises agglomerated metal particles.

38. The method according to claim 34, wherein the porous three dimensional matrix comprises metal particles in a polymer matrix.

39. The method according to claim 34, wherein the metal particles are on the surface of the porous substrate or the metal particles are embedded in the porous substrate.

40. The method according to claim 34, wherein the porous three dimensional matrix filters molecules according to size.
